(19) United States
(12) Patent Application Publication
(10) Pub. No.: US 2005/0148535 A1
(43) Pub. Date: Jul. 7, 2005

(54) IAP nucleobase oligomers and oligomeric complexes and uses thereof

(76) Inventors: Eric LaCasse, Ottawa (CA); Daniel McManus, Ottawa (CA)

Correspondence Address: CLARK & ELBING LLP, 101 FEDERAL STREET, BOSTON, MA, 02110, US

(21) Appl. No.: 10/975,974

(22) Filed: Oct. 28, 2004

Related U.S. Application Data
(60) Provisional application No. 60/516,192, filed on Oct. 30, 2003.

(51) Int. Cl. A61K 48/00; C07H 21/04; C12N 15/85

(52) U.S. Cl. 514/44; 536/23.2; 435/455; 435/320.1

(57) ABSTRACT

The present invention provides nucleobase oligomers and oligomer complexes that inhibit expression of an IAP polypeptide, and methods for using them to induce apoptosis in a cell. The nucleobase oligomers and oligomer complexes of the present invention may also be used to form pharmaceutical compositions. The invention also features methods for enhancing apoptosis in a cell by administering a nucleobase oligomer or oligomer complex of the invention in combination with a chemotherapeutic or chemosensitizing agent.

### CROSS-REFERENCE TO RELATED APPLICATIONS

This application claims benefit from U.S. Provisional Application No. 60/516,192, filed Oct. 30, 2003, hereby incorporated by reference.

### BACKGROUND OF THE INVENTION The invention relates to IAP nucleobase oligomers and oligomeric complexes and methods of using them to induce apoptosis.

One way by which cells die is referred to as apoptosis, or programmed cell death. Apoptosis often occurs as a normal part of the development and maintenance of healthy tissues. The process may occur so rapidly that it is difficult to detect.

The apoptosis pathway is now known to play a critical role in embryonic development, viral pathogenesis, cancer, autoimmune disorders, and neurodegenerative diseases, as well as other events. The failure of an apoptotic response has been implicated in the development of cancer, autoimmune disorders, such as lupus erythematosis and multiple sclerosis, and in viral infections, including those associated with herpes virus, poxvirus, and adenovirus.

The importance of apoptosis in cancer has become clear in recent years. The identification of growth promoting oncogenes in the late 1970's gave rise to an almost universal focus on cellular proliferation that dominated research in cancer biology for many years. Long-standing dogma held that anti-cancer therapies preferentially targeted rapidly dividing cancer cells relative to “normal” cells. This explanation was not entirely satisfactory, since some slow growing tumors are easily treated, while many rapidly dividing tumor types are extremely resistant to anti-cancer therapies. Progress in the cancer field has now led to a new paradigm in cancer biology wherein neoplasia is viewed as a failure to execute normal pathways of programmed cell death. Normal cells receive continuous feedback from their neighbors through various growth factors, and commit “suicide” if removed from this context. Cancer cells somehow bypass these commands and continue inappropriate proliferation. It is now believed that many cancer therapies, including radiation and many forms of chemotherapy, previously thought to act by causing cellular injury, actually work by triggering apoptosis.

Both normal cell types and cancer cell types display a wide range of susceptibility to apoptotic triggers, although the determinants of this resistance are only now under investigation. Many normal cell types undergo temporary growth arrest in response to a sub-lethal dose of radiation or cytotoxic chemical, while cancer cells in the vicinity undergo apoptosis. This differential effect at a given dose provides the crucial treatment window that allows successful anti-cancer therapy. It is therefore not surprising that resistance of tumor cells to apoptosis is emerging as a major category of cancer treatment failure.

Several potent endogenous proteins that inhibit apoptosis have been identified, including the Bcl-2, and IAP protein families in mammals. Certain members of the IAP family directly inhibit terminal effector caspases, i.e., casp-3 and casp-7, engaged in the execution of cell death, as well as the key mitochondrial initiator caspase, casp-9, important to the mediation of cancer chemotherapy induced cell death. The IAPs are the only known endogenous caspase inhibitors, and thus play a central role in the regulation of apoptosis.

The IAPs have been postulated to contribute to the development of some cancers, and a postulated causal chromosomal translocation involving one particular IAP (cIAP2/HIAP1) has been identified in MALT lymphoma. A recent correlation between elevated XIAP, poor prognosis, and short survival has been demonstrated in patients with acute myelogenous leukemia. Furthermore, XIAP was highly over-expressed in many tumor cell lines of the NCI panel.

There exists a need for improved cancer therapeutics and, in particular, therapeutics that can induce cancer cells to undergo apoptosis and override anti-apoptotic signals provided in such cells.

### SUMMARY OF THE INVENTION

The invention relates to IAP nucleobase oligomers and oligomeric complexes and methods of using them to induce apoptosis.

In one aspect, the invention generally features a substantially pure nucleobase oligomer containing a duplex containing at least eight but no more than thirty consecutive nucleobases of XIAP (SEQ ID NO: 21), HIAP-1 (SEQ ID NO: 53), or HIAP-2 (SEQ ID NO: 47), where the duplex reduces expression of an IAP. In one embodiment, the duplex contains a first domain containing between 21 and 29 nucleobases and a second domain that hybridizes to the first domain under physiological conditions, where the first and second domains are connected by a single stranded loop. In another embodiment, the loop contains between 6 and 12 nucleobases. In one embodiment, the loop contains 8 nucleobases. The duplex may be selected, for example, from the group consisting of SEQ ID NOs: 32-36, and reduce expression of XIAP. In another preferred embodiment, the duplex is selected from the group consisting of SEQ ID NOs: 42-46, and reduces expression of HIAP-2.

In another aspect, the invention features a nucleobase oligomeric complex containing paired sense and antisense strands, where the complex contains at least eight, but no more than thirty, consecutive nucleobases corresponding to a sequence of any one of XIAP (SEQ ID NO: 21), HIAP-1 (SEQ ID NO: 53), or HIAP-2 (SEQ ID NO: 47), and the complex reduces expression of an IAP. In one embodiment, the complex contains any one of SEQ ID NOs: 1-31, 37-41, and 54-65. In another embodiment, the nucleic acid molecule is dsRNA. In another embodiment, the complex contains at least one or two modifications (e.g., a modified sugar, nucleobase, or internucleoside linkage). In another embodiment, the modification is a modified internucleoside linkage selected from the group consisting of phosphorothioate, methylphosphonate, phosphotriester, phosphorodithioate, and phosphoselenate linkages. In still other embodiments, the complex contains at least one modified sugar moiety (e.g., a 2′-O-methyl group or a 2′-O-methoxyethyl group). In another embodiment, the complex contains at least one modified nucleobase (e.g., 5-methyl cytosine, a chimeric nucleobase oligomer, RNA residues, or RNA residues linked together by phosphorothioate linkages).

In another aspect, the invention features an expression vector encodes a nucleobase oligomer or nucleobase oligomeric complex of any one of the previous aspects. In one embodiment, a nucleic acid sequence encoding the nucleobase oligomer or nucleobase oligomeric complex is operably linked to a promoter. In another embodiment, the promoter is the U6 PolIII promoter, or the polymerase III H1 promoter

In another aspect, the invention features a cell containing the expression vector of a previous aspect. In one embodiment, the cell is a transformed human cell that stably expresses the expression vector. In another embodiment, the cell is in vivo. In yet another embodiment, the cell is a human cell (e.g., a neoplastic cell).

By “biological response modifying agent” is meant an agent that stimulates or restores the ability of the immune system to fight disease. Some, but not all, biological response modifying agents may slow the growth of cancer cells and thus are also considered to be chemotherapeutic agents. Examples of biological response modifying agents are interferons (alpha, beta, gamma), interleukin-2, rituximab, and trastuzumab.

By “cell” is meant a single-cellular organism, cell from a multi-cellular organism, or it may be a cell contained in a multi-cellular organism.

By “chemosensitizer” is meant an agent that makes tumor cells more sensitive to the effects of chemotherapy. In one example, TRAIL is a chemosensitizer.

By “chemotherapeutic agent” is meant an agent that is used to kill cancer cells or to slow their growth (e.g., those listed in Table 5). Accordingly, both cytotoxic and cytostatic agents are considered to be chemotherapeutic agents.

By consecutive nucleobases “corresponding to” a reference sequence is meant that the order of the nucleobases are identical to the order of the nucleobases in the reference sequence, irrespective of the backbone or linkages joining the nucleobases.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The antisense nucleic acid may contain a modified backbone, for example, phosphorothioate, phosphorodithioate, or other modified backbones known in the art, or may contain non-natural internucleoside linkages.

By “duplex” is meant a single unit containing paired sense and antisense domains. For example, a duplex “comprising” 29 nucleobases contains 29 nucleobases on each of the paired sense and antisense strands.

By an “effective amount” is meant the amount of a compound (e.g., a nucleobase oligomer) required to ameliorate the symptoms of a disease, inhibit the growth of the target cells, reduce the size or number of tumors, inhibit the expression of an IAP, or enhance apoptosis of target cells, relative to an untreated patient. The effective amount of active compound(s) used to practice the present invention for therapeutic treatment of abnormal proliferation (i.e., cancer) varies depending upon the manner of administration, the age, body weight, and general health of the subject. Ultimately, the attending physician or veterinarian will decide the appropriate amount and dosage regimen. Such amount is referred to as an “effective” amount.

By “enhancing apoptosis” is meant increasing the number of cells that apoptose in a given cell population (e.g., cancer cells, lymphocytes, fibroblasts, or any other cells). It will be appreciated that the degree of apoptosis enhancement provided by an apoptosis-enhancing compound in a given assay will vary, but that one skilled in the art can determine the statistically significant change in the level of apoptosis that identifies a nucleobase oligomer that enhances apoptosis otherwise limited by an IAP. Preferably, “enhancing apoptosis” means that the increase in the number of cells undergoing apoptosis is at least 10%, more preferably the increase is 25% or even 50%, and most preferably the increase is at least one-fold, relative to cells not administered a nucleobase oligomer of the invention but otherwise treated in a substantially similar manner. Preferably the sample monitored is a sample of cells that normally undergo insufficient apoptosis (i.e., cancer cells). Methods for detecting changes in the level of apoptosis (i.e., enhancement or reduction) are described herein.

By “hybridize” is meant pair to form a duplex or double-stranded complex containing complementary paired nucleobase sequences, or portions thereof. Preferably, hybridization occurs under physiological conditions, or under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “IAP biological activity” is meant any activity known to be caused in vivo or in vitro by an IAP polypeptide.

By “IAP gene” is meant a gene encoding a polypeptide having at least one BIR domain that is capable of modulating (inhibiting or enhancing) apoptosis in a cell or tissue when provided by other intracellular or extracellular delivery methods (see, e.g., U.S. Pat. No. 5,919,912). In preferred embodiments, the IAP gene is a gene having about 50% or greater nucleotide sequence identity (e.g., at least 85%, 90%, or 95%) to at least one of human or murine XIAP, HIAP1, or HIAP2 (each of which is described in U.S. Pat. No. 6,156,535). Preferably the region of sequence over which identity is measured is a region encoding at least one BIR domain and a ring zinc finger domain. Mammalian IAP genes include nucleotide sequences isolated from any mammalian source. Preferably the mammal is a human.

By “IAP protein” or “IAP polypeptide” is meant a polypeptide, or fragment thereof, encoded by an IAP gene.

By “isolated polynucleotide” is meant a nucleic acid (e.g., a DNA) that is free of the genes, which, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “lymphoproliferative disorder” is meant a disorder in which there is abnormal proliferation of cells of the lymphatic system (e.g., T-cells and B-cells).

By a “nucleobase oligomer” is meant any chain of nucleic acids or nucleic acid mimetics.

By a “nucleobase oligomer complex” is meant a pair of antisense and sense nucleobase oligomers.

By a nucleobase oligomer that “reduces the expression” of a target protein (e.g., an IAP polypeptide) is meant one that decreases the amount of a target protein by at least about 5%, more desirable by at least about 10%, 25%, or even 50%, relative to an untreated control. Methods for measuring protein levels are well-known in the art; exemplary methods are described herein. Preferably, a nucleobase oligomer of the invention is capable of enhancing apoptosis and/or decreasing IAP protein levels when present in a cell that normally does not undergo sufficient apoptosis. Preferably the increase is by at least 10%, relative to a control, more preferably 25%, and most preferably 1-fold or more. Preferably a nucleobase oligomer of the invention includes from about 8 to 30 nucleobases. A nucleobase oligomer of the invention may also contain, for example, an additional 20, 40, 60, 85, 120, or more consecutive nucleobases that are complementary to an IAP polynucleotide. The nucleobase oligomer (or a portion thereof) may contain a modified backbone. Phosphorothioate, phosphorodithioate, and other modified backbones are known in the art. The nucleobase oligomer may also contain one or more non-natural linkages.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “portion” is meant a fragment of a protein or nucleic acid that is substantially identical to a reference protein or nucleic acid, and retains at least 50% or 75%, more preferably 80%, 90%, or 95%, or even 99% of the biological activity of the reference protein or nucleic acid using a assay as described herein.

By “positioned for expression” is meant that the polynucleotide of the invention (e.g., a DNA molecule) is positioned adjacent to a DNA sequence which directs transcription and translation of the sequence (i.e., facilitates the production of, for example, a recombinant polypeptide of the invention, or an RNA molecule).

By “promoter” is meant a polynucleotide sufficient to direct transcription. 3′ regions of the native gene. For example, any polynucleotide region upstream of a gene or a region of an mRNA that is sufficient to direct gene transcription.

By “polypeptide” is meant any chain of amino acids, regardless of length or post-translational modification (for example, glycosylation or phosphorylation).

By “proliferative disease” is meant a disease that is caused by or results in inappropriately high levels of cell division, inappropriately low levels of apoptosis, or both. For example, cancer is an example of a proliferative disease. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

By “siRNA” is meant a double stranded RNA comprising a region complementary to an mRNA. Optimally, an siRNA is 17, 18, 19, 20, 21, 22, 23, 24, 25, 26, 27, 28, 29, or 30 nucleotides in length and, optionally, has a two base overhang at one of its 3′ end. siRNAs can be introduced to an individual cell, tissue, organ, or to a whole animals. Most preferably, an siRNA is between 21 and 29 nucleotides in length. siRNAs may be introduced systemically via the bloodstream. Such siRNAs are used to downregulate mRNA levels or promoter activity. Desirably, the siRNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages.

By “shRNA” is meant an RNA comprising a duplex region complementary to an mRNA. For example, a short hairpin RNA (shRNA) may comprise a duplex region containing nucleoside bases, where the duplex is between 19 and 29 bases in length, and the strands are separated by a single-stranded 4, 5, 6, 7, 8, 9, or 10 base linker region. Optimally, the linker region is 6-8 bases in length.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA, which is inserted by artifice into a cell and typically becomes part of the genome of the organism that develops from that cell. Such a transgene may include a gene that is partly or entirely heterologous (i.e., foreign) to the transgenic organism, or may represent a gene homologous to an endogenous gene of the organism.

By “transgenic” is meant any cell that includes a DNA sequence that is inserted by artifice into a cell and becomes part of the genome of the organism that develops from that cell. As used herein, transgenic organisms may be either transgenic vertebrates, such as domestic mammals (e.g., sheep, cow, goat, or horse), mice, or rats, transgenic invertebrates, such as insects or nematodes, or transgenic plants.

The invention features methods and compositions for inducing apoptosis in a cell. Other features and advantages of the invention will be apparent from the detailed description, and from the claims.

### BRIEF DESCRIPTION OF THE DRAWINGS

FIG. 1 shows the nucleic acid sequence of the XIAP coding region SEQ ID NO: 21. The location often siRNA target sequences (SEQ ID NOs: 22-31) is indicated by bold or underline.

FIG. 2 shows the sense sequences for five shRNAs that target XIAP (SEQ ID NOs: 32-36).

FIG. 3 shows the nucleic acid sequence of the XIAP coding region. The location of five target sequences (SEQ ID NOs: 37-41) of the shRNAs shown in FIG. 2 is indicated. RNAi sequences targeting nucleic acid sequences 5 and 2 are-most preferred and have been successfully used to silence XIAP expression. The five RNAi sequences were sequenced and then transiently transfected into HeLa cells. Twenty-four hours after transfection, RNA levels were determined using quantitative RT-PCR (TaqMan® analysis). shRNAs directed against sequences 2 and 5 reduced XIAP MRNA levels by 85% and 70%, respectively (FIG. 9). Loss of XIAP protein was analyzed at 48 hours after transfection. Transfection conditions were optimized to give at least 90% transfection efficiencies that resulted in approximately 80% loss of XIAP protein by RNAi 2 and 70% by RNAi 5 (FIG. 10). XIAP protein levels were not determined for RNAi sequences 1, 3, and 4. Candidate shRNA target sequences are indicated by underlining (FIG. 3) in reference to the highlighted regions denoting BIR domains 1, 2, and 3.

FIG. 4 shows the sense sequence of five shRNA HIAP-2 target sequences (SEQ ID NOs: 42-46).

FIG. 5 shows the nucleic acid sequence of the HIAP-2 coding region (SEQ ID NO: 47). Five HIAP-2 RNAi target sequences are indicated by underlining (SEQ ID NOs: 48-52). For HIAP-2, RNAi targeting sequence #4 was most successful in silencing HIAP-2 expression. BIR domains 1, 2, and 3 are indicated by highlighting.

FIG. 6 shows the HIAP1 Coding Sequence (SEQ ID NO: 53) that contains nucleotides 449 to 2263, which are present in the mRNA sequence described by Liston et al. (GenBank Accession No: U45878). The underlined sequences (SEQ ID NOs: 54-65) indicate the position of the sequences targeted by shRNAs encoded by RNAi vectors. RNAi that targets sequence 1 (SEQ ID NO: 54) was most successful in silencing HIAP-1, while RNAi targeting sequence 5 (SEQ ID NO: 58) also resulted in a significant reduction in HIAP-1 expression. RNAi against other candidate target sequences did not significantly reduce HIAP-1 expression. While RNAi sequences 11 (SEQ ID NO: 64) and 12 (SEQ ID NO: 65) could potentially target both HIAP-1 and HIAP-2, no reduction in either HIAP-1 or HIAP-2 expression was observed.

FIGS. 7A and 7B are schematic diagrams depicting the generation of short hairpin RNAi (shRNAi) vectors. FIG. 7C is a schematic diagram depicting production of shRNAi transcripts from the U6 PolIII promoter.

FIGS. 8A-8D are photomicrographs of H460 human non-small-cell lung carcinoma cells transfected using a 5′ fluorescein labeled 2×2 test MBO and LipofectAMINE 2000. A microscopic evaluation of transformation efficiencies was carried out 24 hours later. For optimization purposes either constant amounts (1 μl) of LipofectAMINE 2000 were combined with increasing amounts of 2×2 MBO (200 nM—FIG. 10A, 1.2 μM—FIG. 10B) or constant amounts (1 μM) of 2×2 MBO were combined with increasing amounts of LipofectAMINE 2000 (0.6 μl—FIG. 10C, 1.0 μl—FIG. 10D).

FIG. 9A shows the sizes of XIAP RNAi sequence products run on a 2.5% agarose gel.

FIG. 9B is a histogram showing mRNA levels in HeLa cells transiently transfected with the XIAP RNAi sequences shown in FIG. 9A. Clones that significantly knocked down XIAP expression levels are denoted with an asterisk. These corresponded to the large EcoRI inserts in 9A.

FIG. 10A is a western blot showing XIAP protein levels relative to GAPDH protein levels present in transiently transfected HeLa cells.

FIG. 10B is a histogram showing quantitation of XIAP protein levels in transiently transfected HeLa cells. XIAP levels were reduced in cells tranfected with XIAP RNAi clones 2C, 2E, 5E, and 5F, but were not reduced when an empty parental control vector was used for transfection. XIAP levels were also not reduced when a HIAP-1 RNAi vector was used for transfection. Thus, demonstrating that the reductions observed were specific to the XIAP RNAi clones.

FIG. 11A is a western blot showing that XIAP protein levels are reduced in three stably tranfected breast cancer cell line clones. The MDA-MB-23 1 cell line was transfected with linearized DNA, and potential XIAP RNAi clones were amplified and screened by western blot analysis. GAPDH protein levels are included shown for comparison. XIAP levels were reduced significantly in X-G4, X-H3, and X-A4 clones.

FIG. 11B is a histogram showing levels of XIAP protein reduction in clones X-G4, X-H3, and X-A4, which showed reductions of XIAP protein by approximately 90%, 80%, and 40%, respectively.

FIG. 12 is a graph showing that cell survival is reduced in XIAP RNAi stably transfected breast cancer cell lines 16 hours after treatment with increasing amounts of TRAIL (tumor necrosis factor (TNF)-related apoptosis inducing ligand).

FIG. 13 is a western blot depicting the effects of XIAP suppression on death pathway proteins in MDA-MB-231 cells stably transfected with a XIAP RNAi vector and treated with TRAIL and on control cells. Lane 1: U6 E1 (vector control) minus TRAIL; Lane 2: U6 E1, plus TRAIL; Lane 3: X-G4 (XIAP RNAi) minus TRAIL; Lane 4: X-G4 (XIAP RNAi) plus TRAIL. In the plus TRAIL conditions, cells were treated for 7 hours with 10 ng/mL TRAIL prior to harvesting cells for western blot analysis.

FIGS. 14A and 14B are graphs showing the effects of XIAP suppression on the survival of MDA-BM-231 cells stably transfected with a XIAP RNAi vector and treated with taxol (paclitaxel) and taxotere (docetaxel). U6-E1 is the empty U6 promoter vector. X-H3, X-A4, and X-G4 are clones transfected with XIAP RNAi vectors.

### DETAILED DESCRIPTION OF THE INVENTION

The present invention provides nucleobase oligomers and oligomeric complexes that inhibit expression of an IAP, and methods for using them to induce apoptosis in a cell. The nucleobase oligomers and oligomeric complexes of the present invention may also be used to form pharmaceutical compositions. The invention also features methods for enhancing apoptosis in a cell by administering an oligonucleotide of the invention in combination with one or more chemotherapeutic agents such as a cytotoxic agent, cytostatic agent, or biological response modifying agent (e.g., adriamycin, vinorelbine, etoposide, taxol, cisplatin, interferon, interleukin-2, monoclonal antibodies). If desirable, a chemosensitizer (i.e., an agent that makes the proliferating cells more sensitive to the chemotherapy) may also be administered. Any combination of the foregoing agents may also be used to form a pharmaceutical composition. These pharmaceutical compositions may be used to treat a proliferative disease, for example, cancer or a lymphoproliferative disorder, or a symptom of a proliferative disease. For example, a pharmaceutical composition is useful for ex vivo therapy. The compositions of the invention may also be used in combination with radiotherapy for the treatment of cancer or other proliferative disease.

Activation of apoptosis in cancer cells offers novel, and potentially useful approaches to improve patient responses to conventional chemotherapy or radiotherapy. XIAP is the most potent member of the IAP gene family in terms of its ability to directly inhibit caspases and to suppress apoptosis.

IAPs Inhibit Apoptosis

The controlled and ‘normal’ physiological process by which cells die is referred to as apoptosis, or programmed cell death. Apoptosis is distinguished from necrosis, another physiological form of cell death which is considered abnormal, or ‘accidental’, and undesirable because of the secondary tissue damage associated with the inflammatory response provoked in such circumstances. Apoptosis occurs as a normal part of the development and maintenance of healthy tissues. The process occurs in a stochastic fashion and apoptotic cells are rapidly removed, such that it is often difficult to detect the process. Deregulated apoptosis occurs in pathophysiological circumstances such as cancer and neurological disorders, and apoptosis is also the means by which chemotherapy and radiotherapy kill neoplastic cells. The induction of apoptotic pathways leads to the activation of a family of proteases, called caspases (Cysteinyl-active centre proteases or aspartases) that cleave proteins at aspartyl residues within defined substrates. These proteases are the main effectors of apoptosis and are responsible for the generation of the majority of morphological and biochemical characteristics associated with apoptosis (Thornberry and Lazebnik, 1998; Earnshaw et al., 1999). The caspases have endogenous inhibitors, referred to as the IAPs, for inhibitors-of-apoptosis (Deveraux et al., 1997; Roy et al., 1997; Stennicke et al., 2002). The IAPs are characterized by the presence of one to three BIR domains in their N-terminus. BIR motifs are novel zinc-finger folded domains originally described in baculoviruses as suppressing host cell apoptosis (LaCasse et al., 1998; Miller, 1999; Salvesen and Duckett, 2002). Gene knock-out studies have demonstrated the essential role these genes play in yeast, C. elegans, and Drosophila (Fraser et al., 1999; Speliotes et al., 2000; Uren et al., 2000). IAPs are also present in higher organisms where the increased redundancy and complexity of IAPs complicates the elucidation of the individual roles each IAP plays in normal physiology and in disease. Table 1 lists eight human IAPs, which were discovered over the past decade, originating with neuronal apoptosis inhibitory protein (NAIP) (Roy et al., 1995).

TABLE 1

| Human | Gene | Alternative names in | Chromosomal |
|---|---|---|---|
| IAPs | symbol | publications or patents | locus |
| NAIP | birc1 | Birc1e/NAIP5 (mouse), | 5q13.1 |
| | | Birc1a/NAIP1 (mouse) | |
| cIAP1 | birc2 | HIAP2, MIHB | 11q22 |
| cIAP2 | birc3 | HIAP1, API2, MIHC, hITA | 11q22 |
| XIAP | birc4 | hILP, hILP1, MIHA (mouse), | Xq25 |
| | | API3 | |
| survivin | birc5 | TIAP (mouse), MIHD, API4 | 17q25 |
| apollon | birc6 | BRUCE (mouse) | 2p21-2p22 |
| livin | birc7 | KIAP, ML-IAP, cIAP3, HIAP3 | 20q13.3 |
| hILP2 | birc8 | Ts-IAP, TIAP | 19q11.3 |

IAPs have been identified as playing an important role in the development of cancer (LaCasse et al., 1998; Altieri, 2003). Many investigations have found that IAP levels increase in cancer, and have found that patients with increased levels of IAP expression levels are more likely to have a poor prognosis. In addition, gene amplifications involving cIAP1 and cIAP2 (Imoto et al., 2001; Imoto et al., 2002; Dai et al., 2003), as well as a causal translocation involving cIAP2 in marginal zone lymphomas of the MALT (mucosa-associated lymphoid tissue) have been identified (Dierlamm et al., 1999; Liu et al., 2001). XIAP, the most potent of the IAPs, is implicated in cancer by several lines of evidence (Tamm et al., 2000; Holcik and Korneluk, 2001; Liston et al., 2001). Antisense and RNA interference methods offer a promising means of downregulating IAP expression and inducing apoptosis.

Nucleobase Oligomer Approaches To Gene Down-Regulation

Antisense oligonucleotides (ASOs) are synthetic nucleobase oligomers that specifically hybridize with target mRNA transcripts. This hybridization targets the mRNA for degradation by RNAseH recognition of the heteroduplex and degradation of the mRNA. While RNAseH is the principal mechanism of action by which antisense works, inhibition of protein translation and altered intron splicing have also been reported (Agrawal and Kandimalla, 2000). An antisense nucleobase oligomer is a compound that includes a chain of several nucleobases, typically 18-24, joined together by linkage groups. Nucleobase oligomers may contain natural and non-natural oligonucleotides, both modified and unmodified, in addition to modified backbone linkages, such as phosphorothioate and phosphorodiamidate morpholino, oligonucleotide mimetics such as protein nucleic acids (PNA), locked nucleic acids (LNA), and arabinonucleic acids (ANA).

The development of phosphorothioate ODNs provided the ODNs with increased stability against endogenous nucleases. While this increased stability allowed ASOs to be used in clinical settings, the highly polyanionic nature of phosphorothioate ODNs has limited their usefulness. Phosphorothioate ASOs are often referred to as 1^{st} generation ASOs. One such compound is on the market for limited ocular use, while a phosphorothioate ODN targeting bcl-2, Genasense/G3 139, is nearing completion of several Phase 3 clinical trials (Pirollo et al., 2003). Second generation ASOs typically have alkoxy substitutions at the 2′ position of an RNA base, such as 2′O-methyl (Ome) or 2′O-methoxyethyl (MOE), and phosphorothioate DNA residues, in what is termed mixed-backbone oligonucleotides (MBO) or chimeric ASOs. Such ASOs display improved safety and pharmacokinetic profiles in animal models and in humans (Zhou and Agrawal, 1998). The ‘mixture’ of modified RNA and DNA bases is necessitated by the fact that the modified RNA bases do not activate RNAseH once hybridized, while phosphorothioate DNA does. Thus, hybrid molecules with modified RNA bases flanking a core of phosphorothioate DNA residues are effective ASOs. These hybrids are referred to as wingmers, or as 2×2 or 4×4 MBOs, with 2 or 4 flanking 2′O-methyl RNA bases either side, respectively. Several of these second generation MBO compounds are currently in either Phase 1 or Phase 2 trials.

RNAi oligomers are typically RNA duplexes (doubled-stranded RNA or dsRNA) of synthetic complementary monomers of 21-23 nucleotides (nts), with two nucleotide 3′ overhangs each. Alternatively, RNAi oligomers are short hairpin molecules of approximately 50-75 nucleotides with a duplexed region of 21-29 base pairs, which is part of a stem-loop structure that optionally contains 3′ UU-overhangs produced by RNA polymerase III (see Table 2). These molecules are called small interfering RNAs (siRNAs), or short-hairpin RNAs (shRNAs) respectively, and have recently been shown to mediate sequence-specific inhibition of gene expression in mammalian cells via a post-transcriptional gene silencing mechanism termed RNA interference (RNAi) and together are often simply referred to as RNAi (reviewed in Paddison and Hannon, 2002; Dykxhoorn et al., 2003; Shi, 2003). Table 2 provides examples of structures for some of the ASO and RNAi compounds described herein.

TABLE 2

| | |
|---|---|
| Typical | |
| Size (nts | |
| and/or | Composition/ structure |

| | | |
|---|---|---|
| Type of ASO or RNAi | bps) | (using 5′ to 3′ convention) |

| | | |
|---|---|---|
| Minimally-modified ASO | 18-21 | XsXsNoNoNoNoNoNoNoNoNoNoNoNoNoNoXsXs |
| PS ODN (1^{st} generation | 16-25 | NsNsNsNsNsNsNsNsNsNsNsNsNsNsNsNsNsNs |
| ASO) | | |
| MBO (2^{nd} generation | 16-25 | XsXsXsXsNsNsNsNsNsNsNsNsNsNsXsXsXsXs |
| ASO) | | |
| RNAi duplex (siRNA) | 21-23 | XXXXXXXXXXXXXXXXXXXXXdTdT |
| | | dTdTXXXXXXXXXXXXXXXXXXXXX |
| RNAi hairpin (shRNA) | 21-29 | 5′-XXXXXXXXXXXXXXXXXXXXXXXXXXX-X_{]} |
| | | 3′-UUUUXXXXXXXXXXXXXXXXXXXXXXXXXXX-X |

Legend: No, a phosphodiester DNA base consisting of A, G, C, or T;
Ns, a phosphorothioate DNA base consisting of A, G, C, or T (IUPAC codes: F, E, O or Z, respectively);
Xs, a 2′0-methyl RNA nucleoside with a phosphorothioate linkage consisting of A, G, C or U;
X, a natural or synthetic RNA nucleoside consisting of A, G, C or U
dT, deoxythymidine-tail base addition (overhang)
U, uracil-tail base incorporation (overhang)

For expression of shRNAs within cells, plasmid or viral vectors may contain, for example, a promoter, including, but not limited to the polymerase I, II, and III H1, U6, BL, SMK, 7SK, tRNA polIII, tRNA(met)-derived, and T7 promoters, a cloning site for the stem-looped RNA coding insert, and a 4-5-thymidine transcription termination signal. The polymerase III promoters generally have well-defined initiation and stop sites and their transcripts lack poly(A) tails. The termination signal for these promoters is defined by the poly-thymidine tract, and the transcript is typically cleaved after the second uridine. Cleavage at this position generates a 3′ UU overhang in the expressed shRNA, which is similar to the 3′ overhangs of synthetic siRNAs.
RNAi Sequence Selection And Identification

Methods of RNAi are gaining widespread acceptance and use, and may eventually replace antisense as a validation tool. One approach to RNAi involves the use of chemically-synthesized duplexes of RNA (natural, or more preferably, modified-bases for increased stability), termed siRNA. These duplexes are transfected in much the same way that ASOs are, and the screening optimization process is described above. While some of the principles that make antisense work, such as an open RNA structure, may equally apply to RNAi (Bohula et al., 2003), there are clearly many differences that may make an RNAi sequence more effective than an ASO sequence and vice versa (Bertrand et al., 2002; Aoki et al., 2003; Grunweller et al., 2003; Holen et al., 2003; Hough et al., 2003; Vickers et al., 2003; Xu et al., 2003). Highly informative steps for design and selection are given in a review by Dykxhoorn et al. (2003), and other approaches known in the art (Yu et al., 2002; Sohail et al., 2003).

siRNA Design

Preferred siRNAs may be selected using the following criteria.

Target Sequence GC Ratio

First, preferred siRNAs having 21 or 23 nucleotides are selected in the coding region of an mRNA of interest having a GC ratio close to 50%. Optimally, the GC ratio is between 45% and 55%. Less preferred siRNAs have 60% GC content to 70% GC content. Typically, siRNAs having greater than 70% GC content are not preferred, given that they induce decreased levels of gene silencing relative to siRNAs having preferred levels of GC content.

Target Sequence Position

Second, preferred siRNAs are selected from regions that are not within 50-100 nucleotides of an AUG start codon or within 50-100 nucleotides of the termination codon.

Target Sequence Base Content

Third, preferred siRNAs are selected from target sequences that start with two adenosines. When a target sequence starting with AA is selected, siRNA with dTdT overhangs can be produced. Such siRNAs are easier and less expensive to synthesize, and generally show improved resistance to nucleases. In addition, preferably, the targeted region does not contain three or more consecutive guanosines. Such poly-G sequences can hyperstack and form agglomerates that potentially interfere in the siRNA silencing mechanism.

Target Sequence Specificity

Fourth, preferred siRNAs are selected from target sequences that are not homologous to other genes unrelated to IAPs. BLAST searches of prospective target sequences are performed to identify those having low homology to nucleic acid sequences other than the gene of interest. This allows the selection of siRNAs having greater specificity and prevents the silencing of genes having homology to the target sequence.

In sum, most preferred target sequences are 23 nucleotides in length, are within the coding sequence of a gene of interest, start with AA, have 50% GC content, are not within 50-100 nucleotides of a start or termination codon, and are not homologous to non-IAP genes. Less preferably, target sequences are 23 nucleotides in length, are in a region of the coding sequence with a GC content between 45 and 55%, do not contain more than three consecutive guanosines, and are not homologous to non-IAP genes. When target sequences that meet all of these criteria are used for siRNA target sequence design, RNAi effectively silences more than 80% of target genes. The rate of success can be further improved by selecting at least two target sequences for siRNA design.

RNAi Target Selection And Identification

While various parameters are used to identify promising RNAi targets, the most effective siRNA and shRNA candidate sequences are identified by empirical testing. One strategy for such testing is to construct a large library of non-overlapping synthetic siRNAs or shRNA encoding vectors that give good coverage of an IAP gene of interest (e.g., XIAP, HIAP-1, or HIAP-2), according to its largest sequenced cDNA, which includes partial 5′ and 3′ UTR sequences. Provided with knowledge of the intron-exon structure of an IAP and with sensitive means of measuring target knock-down, such as Taqman quantitative RT-PCR and ELISA assays, the process of siRNA or shRNA selection is relatively straightforward once conditions have been optimized for transfection and target measurements. In addition to the selections methods described herein other selection strategies exist and are described, for example, by Xu et al. Biochem Biophys Res Commun. 306: 712-717, 2003; Zhang et al., Nucleic Acids Res. 31: e72, 2003; and Sommer et al. Oncogene. 22: 4266-4280, 2003.

XIAP RNAi Target Sequences

Shown in Table 3 are exemplary XIAP RNAi target sequences (SEQ ID NOs: 1-20).

TABLE 3

SEQ ID

| | | | | |
|---|---|---|---|---|
| Sequence | NO: | Offset | GC Ratio | Rank |

| | | | | |
|---|---|---|---|---|
| AACAAGGAGCAGCTTGCAAGA | 1 | 837 | 47.6 | 1 |
| AACACAGGCGACACTTTCCTA | 2 | 655 | 47.6 | 2 |
| AAGGAGCAGCTTGCAAGAGCT | 3 | 840 | 52.4 | 3 |
| AACCTTGTGATCGTGCCTGGT | 4 | 631 | 52.4 | 4 |
| AAAGTGCTTTCACTGTGGAGG | 5 | 893 | 47.6 | 5 |
| AAGTGCTTTCACTGTGGAGGA | 6 | 894 | 47.6 | 6 |
| AACTGATTGGAAGCCCAGTGA | 7 | 920 | 47.6 | 7 |
| AAGTGCTGGACTCTACTACAC | 8 | 554 | 47.6 | 8 |
| AAGACCCTTGGGAACAACATG | 9 | 940 | 47.6 | 9 |
| AAGAGAGTTAGCAAGTGCTGG | 10 | 542 | 47.6 | 10 |
| AACTGGCCAGACTATGCTCAC | 11 | 513 | 52.4 | 11 |
| AAGTCCTTTCAGAACTGGCCA | 12 | 501 | 47.6 | 12 |
| AAAGTCCTTTCAGAACTGGCC | 13 | 500 | 47.6 | 13 |
| AATAGTGCCACGCAGTCTACA | 14 | 297 | 47.6 | 14 |
| AAATAGTGCCACGCAGTCTAC | 15 | 296 | 47.6 | 15 |
| AAGTGGTAGTCCTGTTTCAGC | 16 | 110 | 47.6 | 16 |
| AAGCAGTTGACAAGTGTCCCA | 17 | 1426 | 47.6 | 17 |
| AAGTGTCCCATGTGCTACACA | 18 | 1437 | 47.6 | 18 |
| TCCAAGAAATCCATCCATGGC | 19 | 767 | 47.6 | 19 |
| CCAAGAAATCCATCCATGGCA | 20 | 768 | 47.6 | 20 |

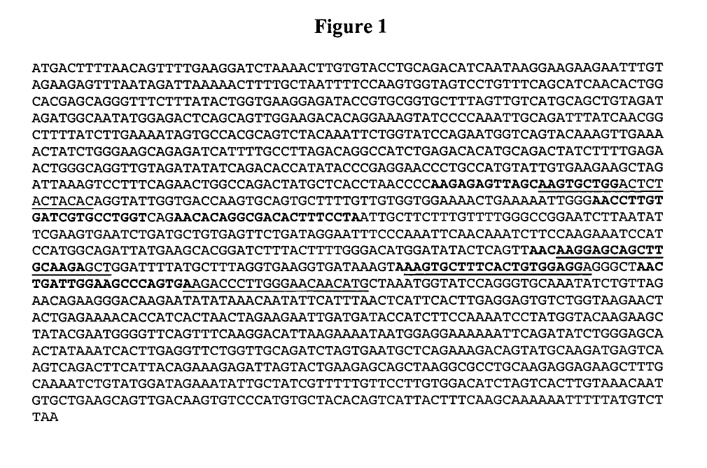

The location of ten of these target sequences relative to the XIAP coding region (SEQ ID NO: 21) is shown in FIG. 1. It is advisable to use modified RNA residues to increase nuclease resistance. Some guidance is given in Hamada et al. (2002); Braasch et al. (2003); and Czaudema et al. (2003).
Stable RNAi

RNAi may also be carried out by stably transfecting cells with a vector that encodes an inhibitory nucleobase oligomer (e.g., siRNA or shRNA). One approach involves the production of shRNA transcripts from a polIII promoter such as H1 (used in the pSUPER vectors, for example; Brummelkamp et al., 2002) and U6 (used in the PCR ‘shagging’ approach of Paddison and Hannon (2002) and Paddison et al. (2002a). This molecular biology approach to generating an RNAi duplex molecule has certain advantages that make it attractive. First, the use and introduction of polIII promoter RNAi vectors into cells allows for the sustained production of RNAi transcripts. Second, the use of retroviral or adenoviral RNAi vectors overcomes limitations relating to plasmid transfection efficiency. Third, polIII RNAi vectors allow for the creation of stable cell lines and transgenic animals (Barton and Medzhitov, 2002; Brummelkamp et al., 2002; Carmell et al., 2003; Hemann et al., 2003; Kunath et al., 2003; Paddison et al., 2002b; Rubinson et al., 2003; Stein et al., 2003; Stewart et al., 2003; Tiscornia et al., 2003), which recapitulate a loss-of-function or null phenotype. Such cells and animals are generated more easily than are genetic knock-out cell lines or animals. The molecular biology approaches described herein are useful in carrying out phenotypic screens of large libraries of gene specific RNAi. In addition, polIII vectors employing the Tet-repressor allow for antibiotic regulation of RNAi production in cells or animals (van de Wetering et al., 2003; Wang et al., 2003).

Full-length RNAi, while useful in C. elegans and Drosophila, presents difficulties when used in mammals because of PKR activation. PKR activation results when dsRNA activates interferon or protein kinase R (PKR) pathways, just as double-stranded RNA viruses do. Activation of PKR shuts down protein synthesis, and can induce cell death. Activation of interferon can also lead to cell death. Because RNAi molecules having fewer than 31 duplexed nucleotides do not activate PKR, RNAi vectors encoding duplexes of no more than 29 nucleotides (Paddison and Hannon, 2002) are preferred.

RNAi Hairpin Sequences (shRNA) and polIII Vector Design

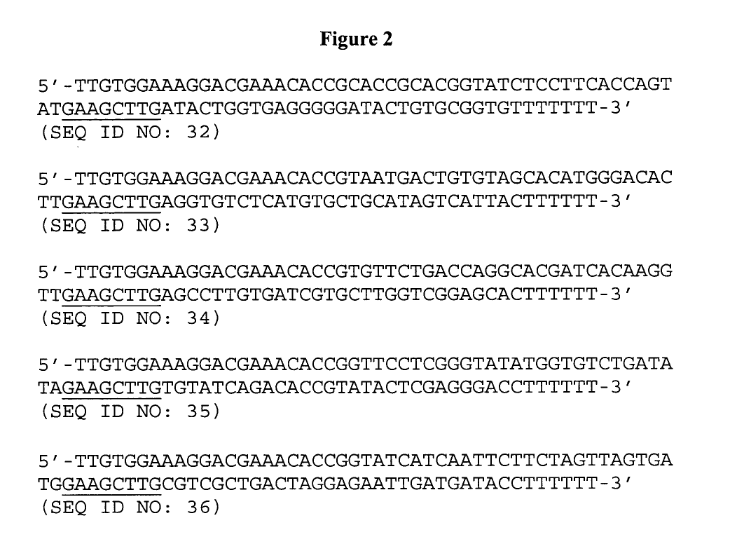

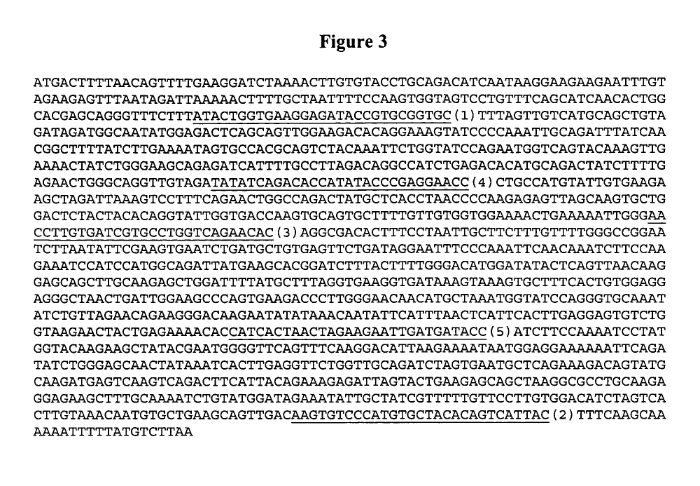

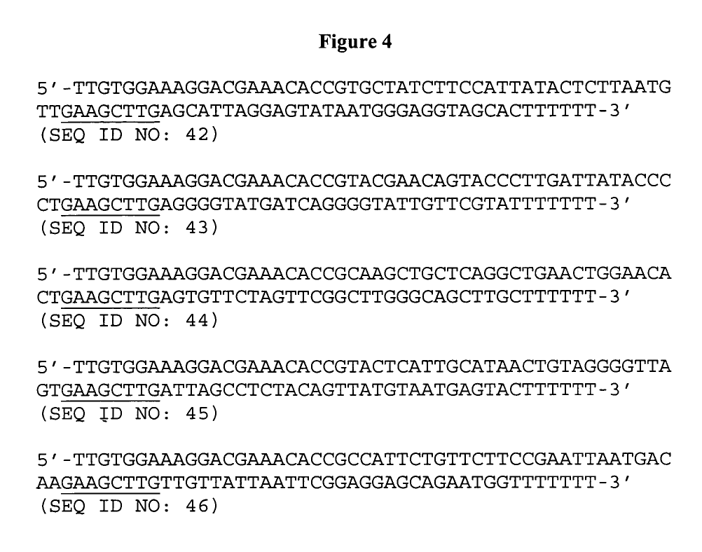

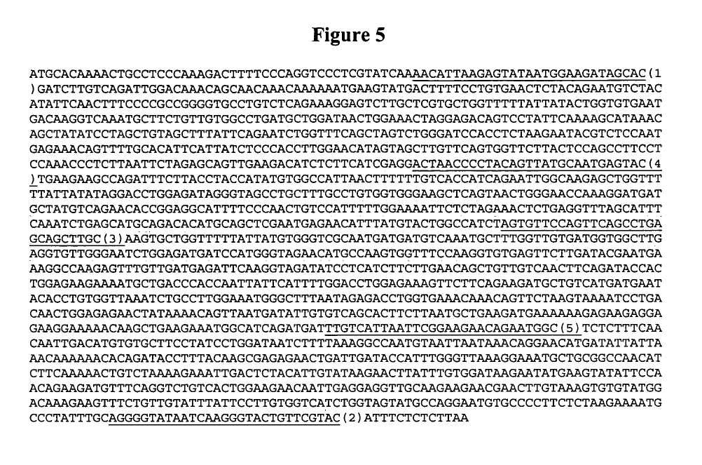

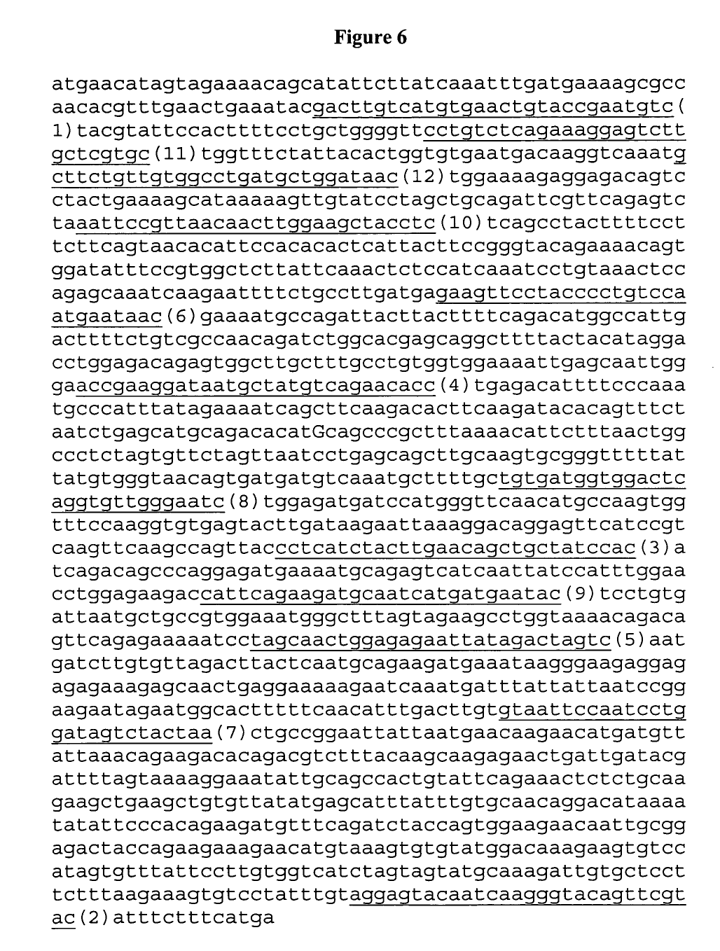

One approach used to identify shRNAs and polIII vectors was described by Paddison and Hannon (Paddison et al., 2002a), and is referred to as the PCR-Shagging method. Protocol details and sequence selection tools are known in the art. Exemplary shRNA sequences that target XIAP are shown in FIG. 2 and the location of the shRNA target sequences in the XIAP coding region is shown in FIG. 3; exemplary shRNA sequences that target HIAP-2 are shown in FIG. 4, and the location of the shRNA target sequences in the HIAP-2 coding region is shown in FIG. 5. Exemplary shRNA sequences target HIAP-1 within the HIAP-1 coding region, as shown in FIG. 6.

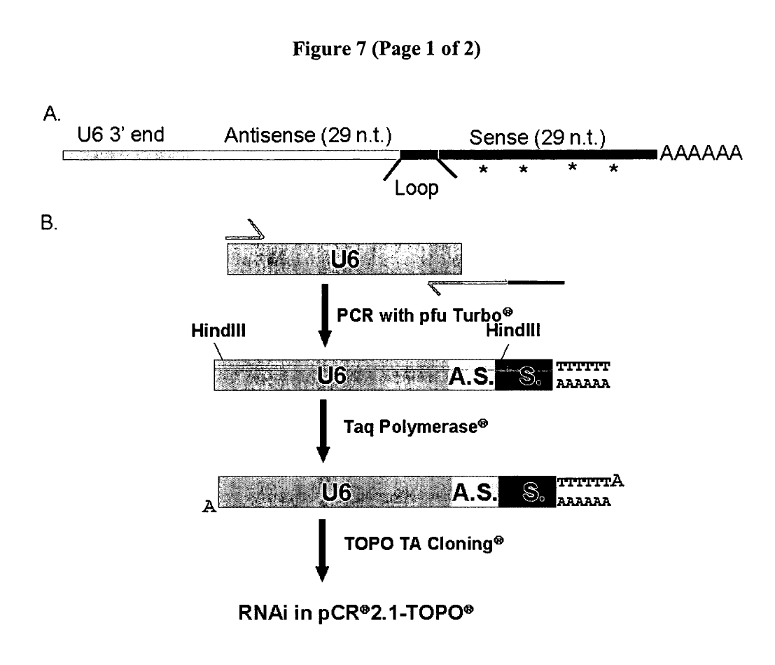

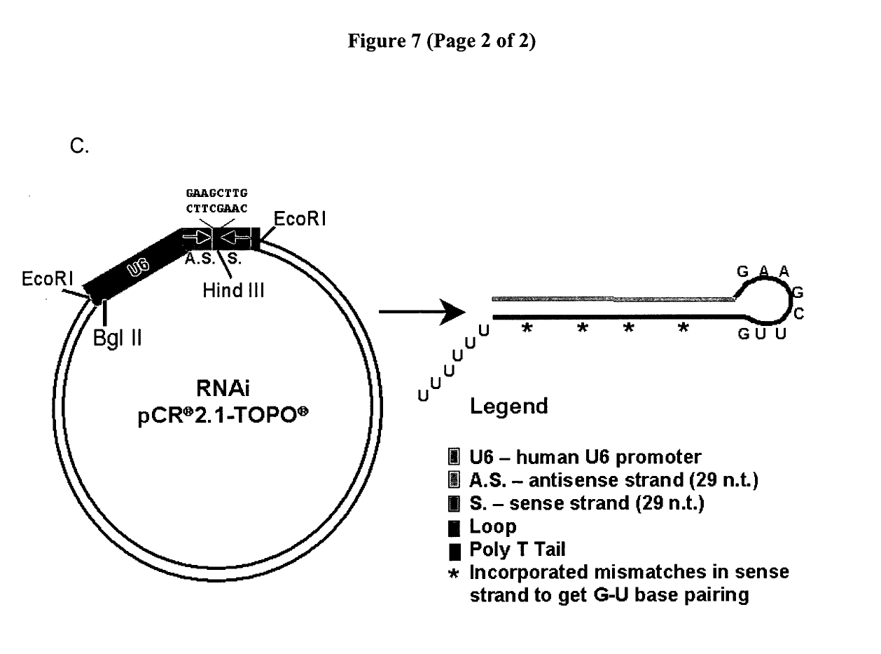

In an exemplary method used in the generation of RNAi vectors, the human U6 snRNA polIII promoter is used to produce a short RNA transcript that is designed for RNAi purposes to form a stem-loop structure. The strategy maintains the U6 transcript initiating ‘G’ residue, and hence all RNAi transcripts will start with ‘G.’ This will restrict the RNAi target sequence to those than contain a ‘C’ at the 3′ position of the sense strand. The transcript is terminated by a run of Ts that are incorporated at the end of the hairpin by the PCR primer. Paddison and Hannon have found that hairpins having 27-29 nucleotides in the duplex, or stem, are more effective than those with 19-21 nucleotide stems. Desirably, a few G-U base pairings are included in the sense strand of the stem, which are permitted in dsRNA alpha helices. These G-U base pairings stabilize hairpins during bacterial propagation. A PCR-based approach allows the rapid generation of multiple different RNAi sequences by incorporating the sequences in a large PCR primer of approximately 93 nucleotides, of which 21 nucleotides are to be used for amplification of the U6 promoter. The final PCR product is then subdloned using TOPO TA Cloning (Invitrogen, San Diego, Calif.), which allows method polymerase chain reaction products to be rapidly cloned into plasmid vectors. DNA from the TOPO clone, which contains the RNAi cassette with its own promoter, can readily be excised and subcloned into numerous other vectors. The actual hairpin PCR primer is the reverse complement with respect to the intended transcript, onto which is added 21 nucleotide homology to the U6 promoter. FIG. 7 illustrates the final shRNA plasmid vector and the predicted hairpin transcript to be generated. HPLC or SDS-PAGE purification of the large primer is not necessary as this limits yield and increases cost. In the end, the analytical and functional screens, as well as DNA sequencing, will verify the integrity of the RNAi vector sequence. A plasmid encoding approximately 300 nucleotides of the U6 promoter is used as a template. PCR conditions may include 4% DMSO to destroy secondary structures induced by the hairpin. Preferably, diagnostic restrictions sites are incorporated to aid in clone selection and verification.

Optimization of Experimental Conditions

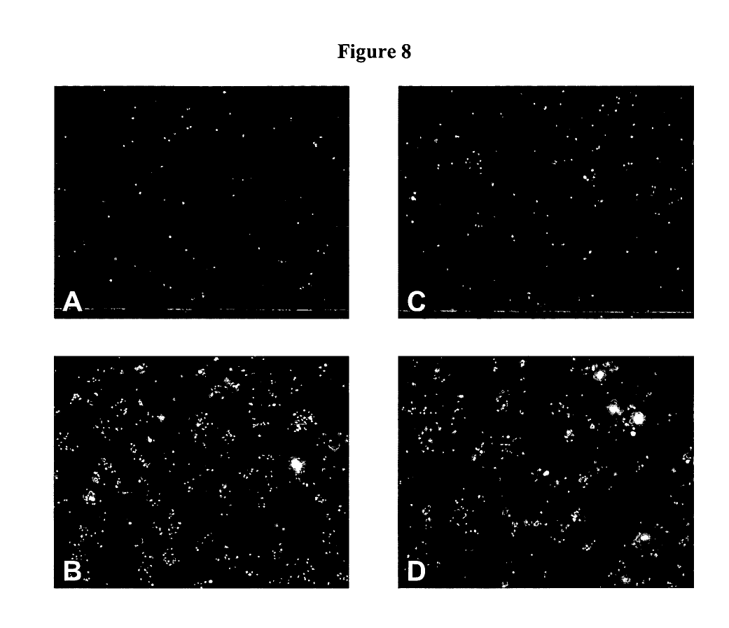

Prior to RNAi target selection, transfection conditions are established in cell lines that are predicted to have high transfection efficiencies and measurable target levels, e.g., using fluorescently-tagged oligomers. While many methods of cell transfection exist (e.g. calcium phosphate, DEAE-dextran, electroporation), the development of highly efficient liposomal transfection agents with reduced cytotoxicity lends themselves to RNAi screening (e.g. Lipofectin, LipofectAMINE PLUS, LipofectAMINE 2000). This is especially useful when RNAi is used to target IAP genes whose downregulation induce apoptosis. RNAi effects must clearly be distinguished from non-specific cytotoxicity associated with transfection agents. FIGS. 8A-8D depict optimization results obtained with a liposomal transfection agent, Lipofectamine 2000, used at two different doses and with two different concentrations of ASO. Transfection optimization results with RNAi are expected to parallel those obtained with ASOs. The photomicrographs show that conditions can be discerned which give strong fluorescent-staining for the majority of cells, and in this way multiple conditions and transfection agents can be compared to find the optimal agent and conditions for the cell line in question. More details on this approach can be found in the article by Stein and colleagues (Benimetskaya et al., 2000).

When establishing optimal transfection conditions it may be useful to employ a positive control, such as an shRNA encoding vector that has been used successfully in RNAi. Transfection conditions for this gene are then measured under similar conditions to the ones proposed in the screening strategy. This allows for the optimization of experimental conditions and the identification of some of the technical difficulties associated with the methodology. The knowledge gained from such an exercise is then be applied to the screening process for RNAi against a new target sequence.

Screening Strategies For RNAi Selection

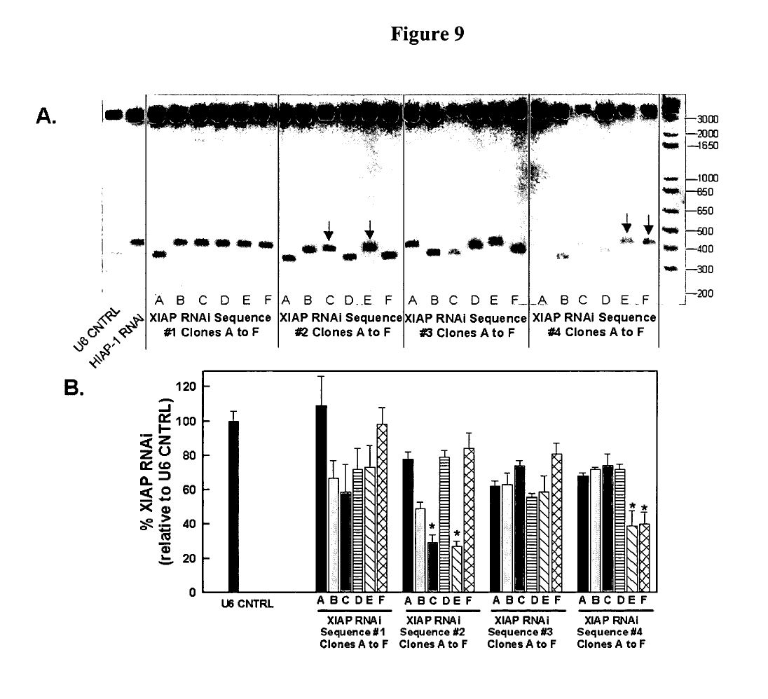

We employed a functional screen to verify shRNA activity by measuring IAP mRNA knock-down post-transfection of plasmid vectors, compared to an empty U6 promoter plasmid. We also employed traditional DNA digest and gel sizing analysis to verify clones and inserts. FIGS. 9A and 9B demonstrate that not all clones of a given PCR reaction will produce a knock-down of XIAP message. The results also show that those clones that do target XIAP efficiently also show the higher molecular weight form on the agarose gel, suggesting that the non-functional clones are likely ‘empty’ U6 promoter constructs, while other large-insert vectors, which show no activity, may not be efficient (e.g., may have wrong insert or the RNAi region may not be accessible to RNAi) RNAi sequences. Positive clones are DNA sequenced to verify their integrity. Our experience has indicated that by this approach we can obtain positive shRNA clones within a short period of time (typically less than two weeks). Thus, this method provides a relatively fast, efficient, and easy approach that is capable of validating candidate RNAi sequences for a large number of genes in a reasonable time frame.

shRNA Sequence Selection

Potential XIAP RNAi clones were screened by digesting with EcoRI and running out the resulting digests on an agarose gel (FIG. 9A). Different size EcoRI fragments were observed, some representing the U6 promoter parental control vector. The clones in FIG. 9A were transiently transfected into HeLa cells, and 24 hours after transfection XIAP MRNA levels were determined by Taqman® analysis. Clones that significant knocked down XIAP expression levels corresponded to the largest inserts identified in FIG. 9A.

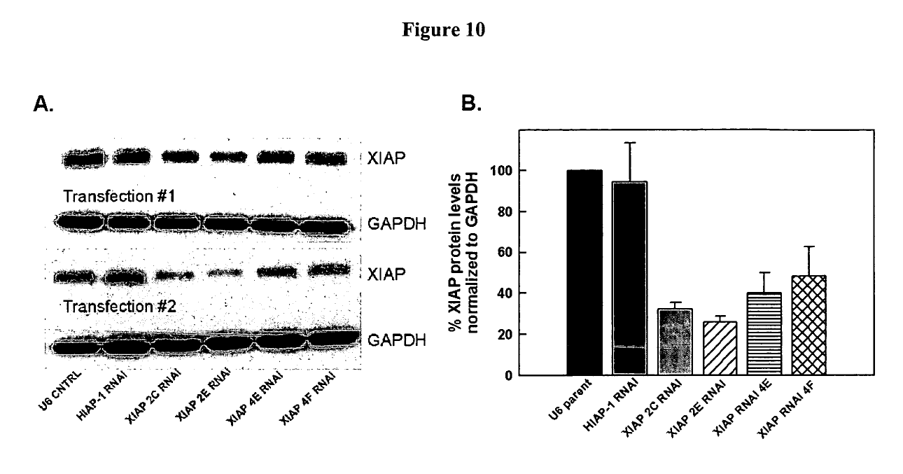

The positive clones identified in FIG. 9A were further validated by transfecting cells with the plasmid shRNA vectors, and measuring for specific XIAP protein down-regulation at 48 hours post-transfection FIG. 9B. FIG. 10A is a western blot showing XIAP protein levels relative to GAPDH levels in HeLa cells transiently transfected with XIAP RNAI pCR® 2.1 TOPO plasmid shows substantial loss of XIAP protein levels relative to cells transfected with the control parental vector. FIG. 10B is a histogram showing densitometry quantitation of XIAP expression in transiently transfected cells. FIG. 10B shows the results for two clones each for two different RNAi sequences identified in FIG. 10. All clones demonstrated activity with one pair slightly outperforming the other. Results with a HIAP-1 RNAi vector demonstrates the specificity of the RNAi for XIAP. The XIAP shRNA results were compared to an shRNA vector for another IAP, HIAP1/cIAP2, and to an empty U6 vector. Clone 2E was sequenced and used for the generation of stable cell lines.

RNAi Controls

In determining the effect of RNAi on a target gene of interest, it is important to use the appropriate controls. Vectors producing shRNA against an irrelevant gene, such as firefly luciferase or the jelly fish GFP are useful controls. Other suitable controls include shRNA-mismatch encoding vectors and vectors that target genes other than the gene of interest to confirm that the RNAi phenotype observed is not related to the effects of expressing shRNA, but rather to the silencing of a gene of interest. Thus, the observed effects are not related to PKR or interferon.

To confirm that the observed phenotype is caused by silencing of a specific target gene, it may be useful to select multiple RNAi constructs that target various sequences within the gene of interest. When multiple siRNAs are used to target a single gene and the same phenotype is induced by each of the different siRNAs, then the phenotype likely results from silencing the specific target gene. One approach for generating an in vitro pool of multiple siRNAs against a full-length mammalian transcript uses the Dicer protein to produce multiple siRNAs against a single target.

Adenoviral or Lentiviral RNAi Vectors

To the molecular biologist, strategies to clone shRNA cassettes into other gene vectors, such as adenovirus and retrovirus, are straightforward. Given that the shRNA cassette may carry its own polIII promoter, sub-cloning strategies need not rely on cloning downstream of a PolIII promoter. Methods for customizing standard shuttle vectors are known to the skilled artisan, and are described by Xia et al. (2002) for adeno-RNAi.

In Vitro Analysis of Gene Function in Transient or Stable Transfection Experiments, or through Viral Transduction

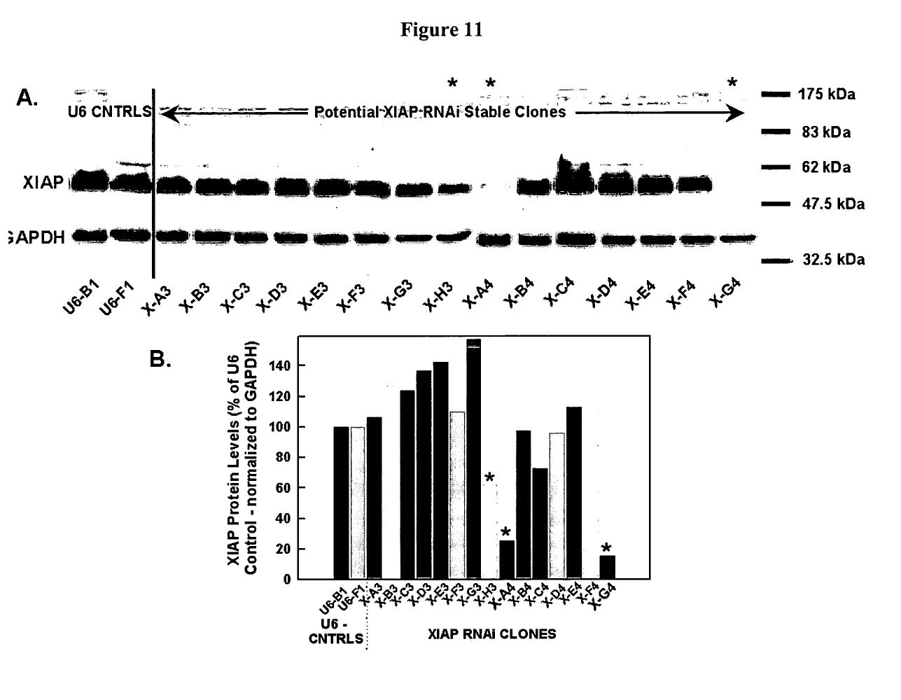

We subcloned a XIAP shRNA cassette (RNAi 2, described in FIG. 3) into pCDNA3 in which the CMV promoter was deleted. This was done so that the new vector would contain a selectable marker (e.g. neomycin resistance) for creating stable cell lines expressing the inhibitory nucleic acids. The breast cancer cell line, MDA-MB-231, was transfected with linearized DNA, and, after recovery, selected in Geneticin/G418 to obtain clonal populations that were screened for XIAP protein knock-down by western blot. FIGS. 11A and 11B demonstrate that of the 15 clones tested, three produced substantial down-regulation of XIAP protein (40, 80, and 90%). Interestingly, the positive clones were actually some of the slower growing colonies. Therefore, it is possible to create stable cell lines and to have continued expression of RNAi molecules in a cell line that is amenable to further study. The creation of RNAi stable cell lines will provide another tool to aid in our analysis of apoptosis control.

XIAP RNAi

Of all IAPs identified to date, XIAP is the most potent inhibitor of apoptosis, and provides the broadest protection against the cytotoxic effects of radiation and chemo- and immuno therapies. It is a key cellular survival factor whose translation is induced under stress. XIAP expression levels are upregulated in the National Cancer Institute 60 human tumor cell line screening panel, a diverse group of human cell lines derived from neoplasms affecting various tissues and organs, including breast, prostate, white blood cells, colon, central nervous system, ovary, skin, kidney, and nonsmall cell lung cancer. XIAP levels are elevated in all the tumor cell lines in the panel relative to XIAP levels in normal liver. XIAP levels are also elevated in pancreatic carcinoma, relative to levels present in surrounding tissue. Given that XIAP levels are increased in a variety of cancers, XIAP is a promising clinical target for RNAi, given that loss of XIAP is a prerequisite for apoptotic cell death.

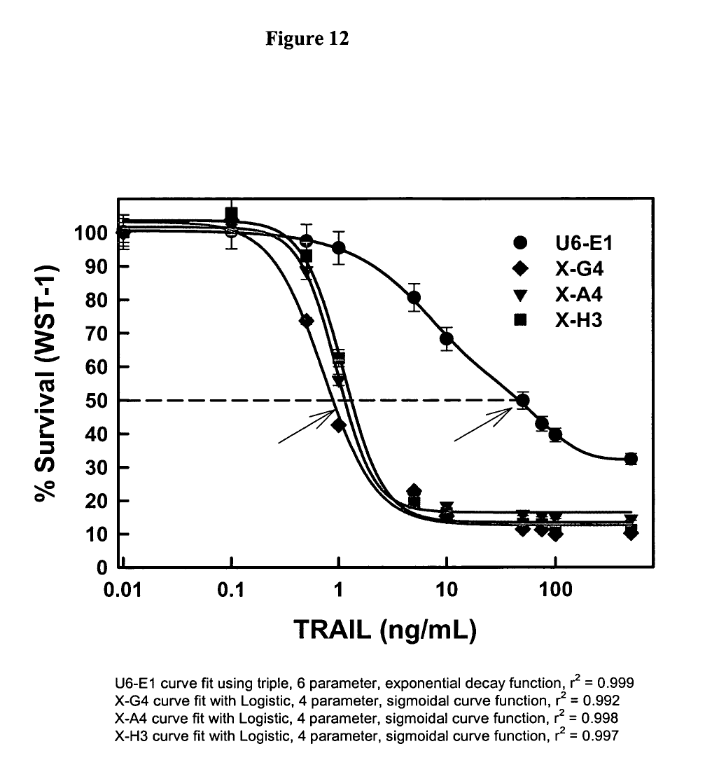

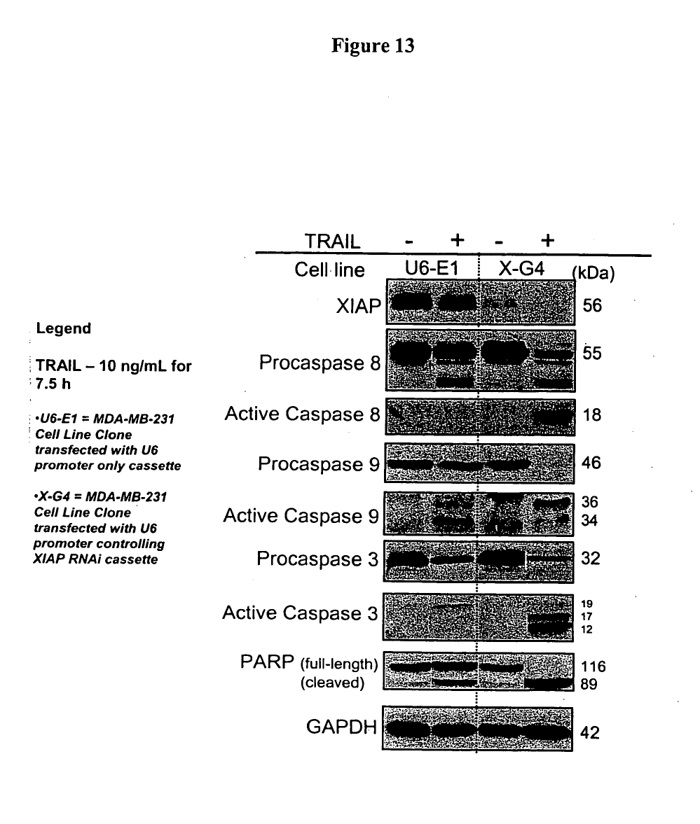

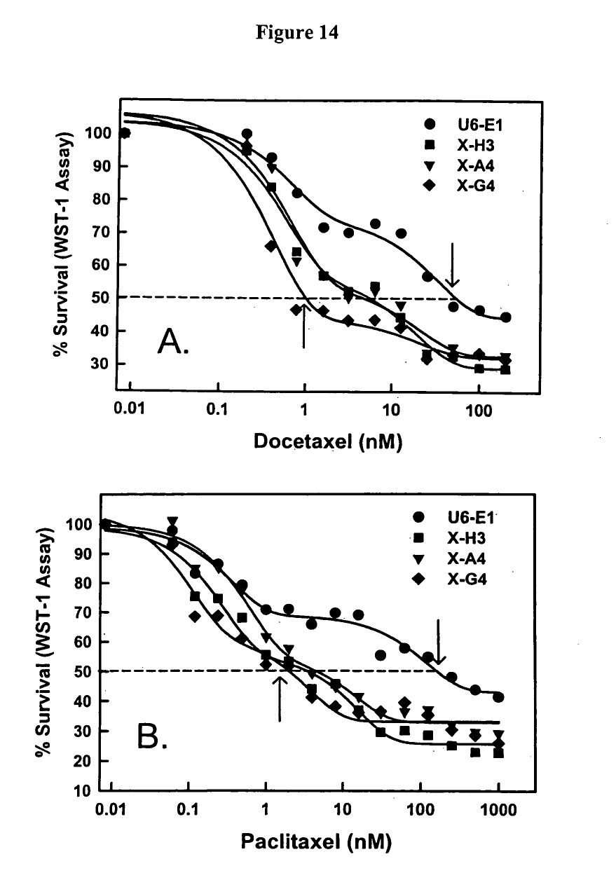

The effect of reducing XIAP levels was evaluated in combination with the administration of another cancer therapeutic, TRAIL (tumor necrosis factor (TNF)-related apoptosis inducing ligand), in a breast cancer cell line, MDA-MB-231, which was stably transfected with a XIAP RNAi vector, as described herein. TRAIL, which is a member of the growing TNF superfamily, induces rapid apoptosis when bound to its receptor. When TRAIL was administered to cell lines stably transfected with a XIAP RNAi vector, cell survival was significantly decreased relative to corresponding control cells transfected with an empty parental vector (FIG. 12). As shown in FIG. 13, levels of cell death mediating proteins were significantly altered following TRAIL treatment of the XIAP RNAi expressing cells relative to control cells. The effect of TRAIL treatment on XIAP RNAi expressing cells is shown in lane 4. Levels of active caspases 3, 8, and 9 were increased in response to TRAIL treatment, while levels of the corresponding inactive enzymes decreased in XIAP RNAi expressing cells relative to control cells. In addition, full length PARP cleavage was increased in XIAP RNAi expressing cells treated with TRAIL relative to control cells (FIG. 13).

The effect of reducing XIAP levels was evaluated in combination with the administration of two chemotherapeutic agents, taxol (docetaxel) and taxotere (paclitaxel), in a breast cancer cell line, MDA-MB-231, which was stably transfected with a XIAP RNAi vector. Cells expressing the XIAP RNAi vector showed a significant reduction in cell survival in response to treatment with chemotherapeutic agents, relative to controls cells transfected with the empty parental vector. Thus, stable RNAi-mediated loss of XIAP in breast cancer cells leads to increased sensitivity to standard chemotherapeutic agents.

In vivo Analysis of RNAi Effects on Tumor Xenografts, or Gene Function in Transgenic Animals

Validated shRNA constructs that successfully target a gene of interest are useful for in vivo testing. For example, in tumor xenograft models cells can be transfected or transduced in vitro, and then implanted into an immunodeficient host to analyze tumor growth effects. Alternatively, sub-cutaneous tumors can be injected in situ with adenoviral shRNA vectors. Systemic administration of siRNAs or shRNAs for xenograft studies can also be used (Lewis et al., 2002; McCaffrey et al., 2002; Song et al., 2003, Sorensen et al., 2003; Zender et al., 2003).

RNAi vectors allow the production of transgenic animals that recapitulate a null phenotype without having to go to the trouble or expense of generating a knock-out (Kunath et al., 2003). In one approach, essential genes are targeted for RNAi “knock down,” which would shut down most, but not all, gene expression. Such an approach might allow the analysis of essential genes whose complete knockout would result in embryonal lethality. In another approach, Tet-inducible RNAi systems would permit fine-tuning of RNAi expression in transgenic animals, allowing the analysis of all genes and splice variants in the mouse genome. Such an approach would likely allow the targeting of one specific transcript versus another. Such RNAi methods are known in the art (Martinez et al., 2002; Wilda et al., 2002; Hemann et al., 2003; Miller et al., 2003), and therefore could provide a powerful tool for gene function analysis in the mouse, or in human cells.

Results and Conclusions

Antisense approaches (e.g. ASO) and RNA interference (e.g. siRNA or shRNA) are useful in validating specific IAPs as clinical targets prior to embarking on small molecule screening programs to treat disorders such as cancer and multiple sclerosis. These approaches are also useful for the development of novel therapeutics.

Approaches for validating the IAPs are briefly summarized in Table 4. ASO and RNAi are but two of the approaches taken. Other approaches include adenoviral delivery of full length antisense, stable cell lines expressing full-length antisense, ribozymes, and triplex-forming oligonucleotides (TFOs).

TABLE 4

| IAP | Approach | Results | References |
|---|---|---|---|
| XIAP | Adenoviral AS | Increased apoptosis | Holcik et al., 2000; Sasaki et |
| | | | al., 2000; Zhang et al., 2000; |
| | | | Asselin et al., 2001a; Asselin |
| | | | et al., 2001b; Li et al., 2001; |
| | | | Xiao et al., 2001; Perrelet et |
| | | | al., 2002 |
| XIAP | ASO | Increased apoptosis | Lin et al., 2001; Troy et al., |
| | | | 2001; Bilim et al., 2003; Hu et |
| | | | al., 2003; Miranda et al., 2003 |
| HIAP1 | ASO | Increased apoptosis | Erl et al., 1999; Gordon et al., |
| | | | 2002 |
| HIAP1 | Full-length AS | Increased apoptosis | Wiese et al., 1999 |
| | (AS RNA) | | |
| HIAP2 | Full-length AS | Increased apoptosis | Spalding et al., 2002 |
| | (AS RNA) | | |
| livin | Full-length AS | Increased apoptosis | Kasof and Gomes, 2001 |
| | (AS RNA) | | |
| apollon | ASO | Increased apoptosis | Chen et al., 1999 |
| survivin | ASO | Increased apoptosis | Olie et al., 2000; Mesri et al., |
| | | | 2001; Xia et al., 2002; Zhou et |
| | | | al., 2002 |
| survivin | ASO | Increased ploidy/ | Li et al., 1999; ; Chen et al., |
| | | multinucleation (and | 2000; Kallio et al., 2001; |
| | | apoptosis in some | Shankar et al., 2001; Chen et |
| | | cases) | al., 2003; Kawamura et al., |
| | | | 2003 |
| survivin | ribozyme | Increased apoptosis | Pennati et al., 2002; Choi et |
| | | | al., 2003; Pennati et al., 2003 |
| survivin | RNAi | Increased ploidy/ | Carvalho et al., 2003; Lens et |
| | | multinucleation (and | al., 2003; Williams et al., 2003 |
| | | apoptosis in some | |
| | | cases) | |
| survivin | TFO | Increased apoptosis | Shen et al., 2003 |
| survivin | Full-length AS | Increased apoptosis | Ambrosini et al., 1998; |
| | | | Grossman et al., 1999a; |
| | | | Grossman et al., 1999b; |
| | | | Kanwar et al., 2001; |
| | | | Yamamoto et al., 2002 |
| survivin | Full-length AS | Increased ploidy/ | Li et al, 1999; Sommer et al., |
| | | multinucleation (and | 2003 |
| | | apoptosis in some | |
| | | cases) | |

Oligonucleotides and Other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. patent application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with an IAP. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O—, S—, or N-alkyl; O—, S—, or N-alkenyl; O—, S— or N--alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O-CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleobase oligomers of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention can be prepared as SATE ((S-acetyl-2-thioethyl) phosphate) derivatives according to the methods disclosed in PCT publication Nos. WO 93/24510 or WO 94/26764.

The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

The present invention also includes pharmaceutical compositions and formulations that include the nucleobase oligomers of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration.

Locked Nucleic Acids

Locked nucleic acids (LNAs) are nucleobase oligomers that can be employed in the present invention. LNAs contain a 2′O, 4′-C methylene bridge that restrict the flexibility of the ribofuranose ring of the nucleotide analog and locks it into the rigid bicyclic N-type conformation. LNAs show improved resistance to certain exo- and endonucleases and activate RNAse H, and can be incorporated into almost any nucleobase oligomer. Moreover, LNA-containing nucleobase oligomers can be prepared using standard phosphoramidite synthesis protocols. Additional details regarding LNAs can be found in PCT publication No. WO 99/14226 and U.S. patent application Publication No. US 2002/0094555 A1, each of which is hereby incorporated by reference.

Arabinonucleic Acids

Arabinonucleic acids (ANAs) can also be employed in methods and reagents of the present invention. ANAs are nucleobase oligomers based on D-arabinose sugars instead of the natural D-2′-deoxyribose sugars. Underivatized ANA analogs have similar binding affinity for RNA as do phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars. The use of ANAs in therapy is discussed, for example, in Damha et al., Nucleosides Nucleotides & Nucleic Acids 20: 429-440, 2001.

Delivery of Nucleobase Oligomers and Oligomeric Complexes

We demonstrate herein that naked oligonucleotides are capable on entering tumor cells and inhibiting IAP expression. Nonetheless, it may be desirable to utilize a formulation that aids in the delivery of oligonucleotides or other nucleobase oligomers or oligomeric complexes to cells (see, e.g., U.S. Pat. Nos. 5,656,611, 5,753,613, 5,785,992, 6,120,798, 6,221,959, 6,346,613, and 6,353,055, each of which is hereby incorporated by reference).

Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place.

As used herein, the terms “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

A nucleobase oligomer of the invention, or other negative regulator of the IAP anti-apoptotic pathway, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, PA, 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for IAP modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleobase oligomer of the invention is likely to depend on such variables as the type and extent of the disorder, the overall health status of the particular patient, the formulation of the compound excipients, and its route of administration.

As described above, if desired, treatment with a nucleobase oligomer of the invention may be combined with therapies for the treatment of proliferative disease, such as radiotherapy, surgery, or chemotherapy. Chemotherapeutic agents that may be administered with an IAP RNAi compound are listed in Table 5.

TABLE 5

| | | |
|---|---|---|
| Alkylating agents | cyclophosphamide | lomustine |
| | busulfan | procarbazine |
| | ifosfamide | altretamine |
| | melphalan | estramustine phosphate |
| | hexamethylmelamine | mechlorethamine |
| | thiotepa | streptozocin |
| | chlorambucil | temozolomide |
| | dacarbazine | semustine. |
| | carmustine | |
| Platinum agents | cisplatin | carboplatinum |
| | oxaliplatin | ZD-0473 (AnorMED) |
| | spiroplatinum, | lobaplatin (Aeterna) |
| | carboxyphthalatoplatinum, | satraplatin (Johnson Matthey) |
| | tetraplatin | BBR-3464 (Hoffmann-La Roche) |
| | ormiplatin | SM-11355 (Sumitomo) |
| | iproplatin | AP-5280 (Access) |
| Antimetabolites | azacytidine | tomudex |
| | gemcitabine | trimetrexate |
| | capecitabine | deoxycoformycin |
| | 5-fluorouracil | fludarabine |
| | floxuridine | pentostatin |
| | 2-chlorodeoxyadenosine | raltitrexed |
| | 6-mercaptopurine | hydroxyurea |
| | 6-thioguanine | decitabine (SuperGen) |
| | cytarabin | clofarabine (Bioenvision) |
| | 2-fluorodeoxy cytidine | irofulven (MGI Pharma) |
| | methotrexate | DMDC (Hoffmann-La Roche) |
| | idatrexate | ethynylcytidine (Taiho) |
| Topoisomerase | amsacrine | rubitecan (SuperGen) |
| inhibitors | epirubicin | exatecan mesylate (Daiichi) |
| | etoposide | quinamed (ChemGenex) |
| | teniposide or mitoxantrone | gimatecan (Sigma-Tau) |
| | irinotecan (CPT-11) | diflomotecan (Beaufour-Ipsen) |
| | 7-ethyl-10-hydroxy-camptothecin | TAS-103 (Taiho) |
| | topotecan | elsamitrucin (Spectrum) |
| | dexrazoxanet (TopoTarget) | J-107088 (Merck & Co) |
| | pixantrone (Novuspharma) | BNP-1350 (BioNumerik) |
| | rebeccamycin analogue (Exelixis) | CKD-602 (Chong Kun Dang) |
| | BBR-3576 (Novuspharma) | KW-2170 (Kyowa Hakko) |
| Antitumor | dactinomycin (actinomycin D) | amonafide |
| antibiotics | doxorubicin (adriamycin) | azonafide |
| | deoxyrubicin | anthrapyrazole |
| | valrubicin | oxantrazole |
| | daunorubicin (daunomycin) | losoxantrone |
| | epirubicin | bleomycin sulfate (blenoxane) |
| | therarubicin | bleomycinic acid |
| | idarubicin | bleomycin A |
| | rubidazone | bleomycin B |
| | plicamycinp | mitomycin C |
| | porfiromycin | MEN-10755 (Menarini) |
| | cyanomorpholinodoxorubicin | GPX-100 (Gem Pharmaceuticals) |
| | mitoxantrone (novantrone) | |
| Antimitotic | paclitaxel | SB 408075 (GlaxoSmithKline) |
| agents | docetaxel | E7010 (Abbott) |
| | colchicine | PG-TXL (Cell Therapeutics) |
| | vinblastine | IDN 5109 (Bayer) |
| | vincristine | A 105972 (Abbott) |
| | vinorelbine | A 204197 (Abbott) |
| | vindesine | LU 223651 (BASF) |
| | dolastatin 10 (NCI) | D 24851 (ASTAMedica) |
| | rhizoxin (Fujisawa) | ER-86526 (Eisai) |
| | mivobulin (Warner-Lambert) | combretastatin A4 (BMS) |
| | cemadotin (BASF) | isohomohalichondrin-B (PharmaMar) |
| | RPR 109881A (Aventis) | ZD 6126 (AstraZeneca) |
| | TXD 258 (Aventis) | PEG-paclitaxel (Enzon) |
| | epothilone B (Novartis) | AZ10992 (Asahi) |
| | T 900607 (Tularik) | IDN-5109 (Indena) |
| | T 138067 (Tularik) | AVLB (Prescient NeuroPharma) |
| | cryptophycin 52 (Eli Lilly) | azaepothilone B (BMS) |
| | vinflunine (Fabre) | BNP-7787 (BioNumerik) |
| | auristatin PE (Teikoku Hormone) | CA-4 prodrug (OXiGENE) |
| | BMS 247550 (BMS) | dolastatin-10 (NIH) |
| | BMS 184476 (BMS) | CA-4 (OXiGENE) |
| | BMS 188797 (BMS) | |
| | taxoprexin (Protarga) | |
| Aromatase | aminoglutethimide | exemestane |
| inhibitors | letrozole | atamestane (BioMedicines) |
| | anastrazole | YM-511 (Yamanouchi) |
| | formestane | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | mafosfamide (Baxter International) |
| | glufosfamide (Baxter International) | apaziquone (Spectrum Pharmaceuticals) |
| | albumin + 32P (Isotope Solutions) | O6 benzyl guanine (Paligent) |
| | thymectacin (NewBiotics) | |
| | edotreotide (Novartis) | |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | prednisone |
| antihormonal | conjugated estrogens | methylprednisolone |
| agents | ethinyl estradiol | prednisolone |
| | chlortrianisen | aminoglutethimide |
| | idenestrol | leuprolide |
| | hydroxyprogesterone caproate | goserelin |
| | medroxyprogesterone | leuporelin |
| | testosterone | bicalutamide |
| | testosterone propionate; fluoxymesterone | flutamide |
| | methyltestosterone | octreotide |
| | diethylstilbestrol | nilutamide |
| | megestrol | mitotane |
| | tamoxifen | P-04 (Novogen) |
| | toremofine | 2-methoxyestradiol (EntreMed) |
| | dexamethasone | arzoxifene (Eli Lilly) |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Tyrosine Kinase | imatinib (Novartis) | kahalide F (PharmaMar) |
| Inhibitors | leflunomide (Sugen/Pharmacia) | CEP-701 (Cephalon) |
| | ZD1839 (AstraZeneca) | CEP-751 (Cephalon) |
| | erlotinib (Oncogene Science) | MLN518 (Millenium) |
| | canertinib (Pfizer) | PKC412 (Novartis) |
| | squalamine (Genaera) | phenoxodiol ( ) |
| | SU5416 (Pharmacia) | trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | EKB-569 (Wyeth) | |

Miscellaneous agents

| | |
|---|---|
| SR-27897 (CCK A inhibitor, Sanofi-Synthelabo) | BCX-1777 (PNP inhibitor, BioCryst) |
| tocladesine (cyclic AMP agonist, Ribapharm) | ranpirnase (ribonuclease stimulant, Alfacell) |
| alvocidib (CDK inhibitor, Aventis) | galarubicin (RNA synthesis inhibitor, Dong-A) |
| CV-247 (COX-2 inhibitor, Ivy Medical) | tirapazamine (reducing agent, SRI International) |
| P54 (COX-2 inhibitor, Phytopharm) | N-acetylcysteine (reducing agent, Zambon) |
| CapCell ™ (CYP450 stimulant, Bavarian Nordic) | R-flurbiprofen (NF-kappaB inhibitor, Encore) |
| GCS-100 (gal3 antagonist, GlycoGenesys) | 3CPA (NF-kappaB inhibitor, Active Biotech) |
| G17DT immunogen (gastrin inhibitor, Aphton) | seocalcitol (vitamin D receptor agonist, Leo) |
| efaproxiral (oxygenator, Allos Therapeutics) | 131-I-TM-601 (DNA antagonist, TransMolecular) |
| PI-88 (heparanase inhibitor, Progen) | eflornithine (ODC inhibitor, ILEX Oncology) |
| tesmilifene (histamine antagonist, YM BioSciences) | minodronic acid (osteoclast inhibitor, Yamanouchi) |
| histamine (histamine H2 receptor agonist, Maxim) | indisulam (p53 stimulant, Eisai) |
| tiazofurin (IMPDH inhibitor, Ribapharm) | aplidine (PPT inhibitor, PharmaMar) |
| cilengitide (integrin antagonist, Merck KGaA) | rituximab (CD20 antibody, Genentech) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| ceflatonin (apoptosis promotor, ChemGenex) | |

For any of the methods of application described above, a nucleobase oligomer of the invention is desirably administered intravenously or is applied to the site of the needed apoptosis event (e.g., by injection).

### OTHER EMBODIMENTS

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

References

Agrawal S, and Kandimalla E R. (2000) Antisense therapeutics: is it as simple as complementary base recognition? Mol Med Today. 6, 72-81.

Ambrosini G, Adida C, Sirugo G, and Altieri D C. (1998) Induction of apoptosis and inhibition of cell proliferation by survivin gene targeting. J Biol Chem 273, 11177-11182.

Aoki Y, Cioca D P, Oidaira H, and Kiyosawa K (2003) RNA interference may be more potent than antisense RNA in human cancer cell lines. Clin Exp Pharmacol Physiol 30, 96-102.

Asselin E, Mills G B, and Tsang B K. (2001a) XIAP regulates Akt activity and caspase-3-dependent cleavage during cisplatin-induced apoptosis in human ovarian epithelial cancer cells. Cancer Res. 61, 1862-1868.

Asselin E, Wang Y, and Tsang B K. (2001b) X-linked inhibitor of apoptosis protein activates the phosphatidylinositol 3-kinase/Akt pathway in rat granulosa cells during follicular development. Endocrinology. 142, 2451-2457.

Barton G M, and Medzhitov R (2002) Retroviral delivery of small interfering RNA into primary cells. Proc Natl Acad Sci USA 99: 14943-14945.

Benimetskaya L, Tonkinson J, and Stein C A. (2000) Determination of cellular internalization of fluoresceinated oligonucleotides. Methods Enzymol. 313, 287-297.

Bertrand J R, Pottier M, Vekris A, Opolon P, Maksimenko A, and Malvy C. (2002) Comparison of antisense oligonucleotides and siRNAs in cell culture and in vivo. Biochem Biophys Res Commun. 296, 1000-1004.

Bilim V, Kasahara T, Hara N, Takahashi K, and Tomita Y. (2003) Role of XIAP in the malignant phenotype of transitional cell cancer (TCC) and therapeutic activity of XIAP antisense oligonucleotides against multidrug-resistant TCC in vitro. Int J Cancer. 103, 29-37.

Bohula E A, Salisbury A J, Sohail M, Playford M P, Riedemann J, Southern E M, and Macaulay V M (2003) The efficacy of small interfering RNAs targeted to the type 1 IGF receptor is influenced by secondary structure in the IGF1R transcript. J Biol Chem (missing vol and page info, epub on February 24).

Braasch D A, Jensen S, Liu Y, Kaur K, Arar K, White M A, and Corey D R. (2003) RNA Interference in Mammalian Cells by Chemically-Modified RNA. Biochemistry. 42, 7967-7975.

Brummelkamp T R, Bernards R, and Agami R. (2002) A system for stable expression of short interfering RNAs in mammalian cells. Science. 296, 550-553.

Brummelkamp T R, Bernards R, and Agami R. (2002) Stable suppression of tumorigenicity by virus-mediated RNA interference. Cancer Cell. 2, 243-247.

Carmell M A, Zhang L, Conklin D S, Hannon G J, and Rosenquist T A. (2003) Germline transmission of RNAi in mice. Nat Struct Biol.10, 91-92.

Carvalho A, Carmena M, Sambade C, Earnshaw W C, and Wheatley S P. (2003) Survivin is required for stable checkpoint activation in taxol-treated HeLa cells. J Cell Sci. 116, 2987-2998.

Chen Z, Naito M, Hori S, Mashima T, Yamori T, and Tsuruo T. (1999) A human IAP-family gene, apollon, expressed in human brain cancer cells. Biochem Biophys Res Commun. 264, 847-854.

Chen J, Wu W, Tahir S K, Kroeger P E, Rosenberg S H, Cowsert L M, Bennett F, Krajewski S, Krajewska M, Welsh K, Reed J C, and Ng S C. (2000) Down-regulation of survivin by antisense oligonucleotides increases apoptosis, inhibits cytokinesis and anchorage-independent growth. Neoplasia. 2, 235-241.

Chen J, Jin S, Tahir S K, Zhang H, Liu X, Sarthy A V, McGonigal T P, Liu Z, Rosenberg S H, and Ng S C. (2003) Survivin enhances Aurora-B kinase activity and localizes Aurora-B in human cells. J Biol Chem. 278, 486-490.

Choi K S, Lee T H, and Jung M H. (2003) Ribozyme-mediated cleavage of the human survivin mRNA and inhibition of antiapoptotic function of survivin in MCF-7 cells. Cancer Gene Ther.10, 87-95.

Czauderna F, Fechtner M, Dames S, Aygun H, Klippel A, Pronk G J, Giese K, and Kaufmann J. (2003) Structural variations and stabilising modifications of synthetic siRNAs in mammalian cells. Nucleic Acids Res.31, 2705-2716.

Dai Z, Zhu W G, Morrison C D, Brena R M, Smiraglia D J, Raval A, Wu Y Z, Rush L J, Ross P, Molina J R, Otterson G A, and Plass C. (2003) A comprehensive search for DNA amplification in lung cancer identifies inhibitors of apoptosis cIAP1 and cIAP2 as candidate oncogenes. Hum Mol Genet. 12, 791-801.

Deveraux Q L, Takahashi R, Salvesen G S, and Reed J C. (1997) X-linked IAP is a direct inhibitor of cell-death proteases. Nature. 388, 300-304.

Dierlamm J, Baens M, Wlodarska I, Stefanova-Ouzounova M, Hernandez J M, Hossfeld D K, De Wolf-Peeters C, Hagemeijer A, Van den Berghe H, and Marynen P. (1999) The apoptosis inhibitor gene AP12 and a novel 18q gene, MLT, are recurrently rearranged in the t(11; 18)(q21 ;q21) associated with mucosa-associated lymphoid tissue lymphomas. Blood. 93, 3601-3609.

Dykxhoorn D M, Novina C D, and Sharp P A (2003) Killing the messenger: short RNAs that silence gene expression. Nature Rev Mol Cell Biol 4, 457-467

Earnshaw W C, Martins L M, and Kaufmann S H. (1999) Mammalian caspases: structure, activation, substrates, and functions during apoptosis. Annu Rev Biochem. 68, 383-424.

Erl W, Hansson G K, de Martin R, Draude G, Weber K S, and Weber C. (1999) Nuclear factor-kappa B regulates induction of apoptosis and inhibitor of apoptosis protein-I expression in vascular smooth muscle cells. Circ Res. 84, 668-677.

Fraser A G, James C, Evan G I, and Hengartner M O. (1999) Caenorhabditis elegans inhibitor of apoptosis protein (IAP) homologue BIR-1 plays a conserved role in cytokinesis. Curr Biol. 9, 292-301.

Gordon G J, Appasani K, Parcells J P, Mukhopadhyay N K, Jaklitsch M T, Richards W G, Sugarbaker D J, and Bueno R. (2002) Inhibitor of apoptosis protein-1 promotes tumor cell survival in mesothelioma. Carcinogenesis. 23, 1017-1024.

Grossman D, McNiff J M, Li F, and Altieri D C. Expression and targeting of the apoptosis inhibitor, survivin, in human melanoma. J Invest Dermatol. 113, 1076-1081.

Grossman D, McNiff J M, Li F, and Altieri D C. (1999) Expression of the apoptosis inhibitor, survivin, in nonmelanoma skin cancer and gene targeting in a keratinocyte cell line. Lab Invest. 79, 1121-1126.

Grunweller A, Wyszko E, Bieber B, Jahnel R, Erdmann V A, and Kurreck J. (2003) Comparison of different antisense strategies in mammalian cells using locked nucleic acids, 2′-O-methyl RNA, phosphorothioates and small interfering RNA. Nucleic Acids Res. 31, 3185-3193.

Hamada M, Ohtsuka T, Kawaida R, Koizumi M, Morita K, Furukawa H, Imanishi T, Miyagishi M, and Taira K. (2002) Effects on RNA interference in gene expression (RNAi) in cultured mammalian cells of mismatches and the introduction of chemical modifications at the 3′-ends of siRNAs. Antisense Nucleic Acid Drug Dev. 12, 301-309.

Hemann M T, Fridman J S, Zilfou J T, Hernando E, Paddison P J, Cordon-Cardo C, Hannon G J, and Lowe S W. (2003) An epi-allelic series of p53 hypomorphs created by stable RNAi produces distinct tumor phenotypes in vivo. Nat Genet. 33, 396-400.

Holcik M, Yeh C, Korneluk R G, and Chow T. (2000) Translational upregulation of X-linked inhibitor of apoptosis (XIAP) increases resistance to radiation induced cell death. Oncogene. 19, 4174-4177.

Holcik M, and Korneluk R G. (2001) XIAP, the guardian angel. Nat Rev Mol Cell Biol. 2, 550-556.

Holen T, Amarzguioui M, Babaie E, and Prydz H (2003) Similar behaviour of single-strand and double-strabd si RNAs suggests they act through a common RNAi pathway. Nucleic Acids Res 31, 2401-2407

Hough S R, Wiederholt K A, Burrier A C, Woolf T M, and Taylor M F. (2003) Why RNAi makes sense. Nat Biotechnol. 21, 731-732

Hu Y, Cherton-Horvat G, Dragowska V, Baird S, Korneluk R G, Durkin J P, Mayer L D, and LaCasse E C. (2003) Antisense Oligonucleotides Targeting XIAP Induce Apoptosis and Enhance Chemotherapeutic Activity against Human Lung Cancer Cells in Vitro and in Vivo. Clin Cancer Res. 9, 2826-36.

Imoto I, Yang Z Q, Pimkhaokham A, Tsuda H, Shimada Y, Imamura M, Ohki M, and Inazawa J. (2001) Identification of cIAP1 as a candidate target gene within an amplicon at 11q22 in esophageal squamous cell carcinomas. Cancer Res. 61, 6629-6634.

Imoto I, Tsuda H, Hirasawa A, Miura M, Sakamoto M, Hirohashi S, and Inazawa J. (2002) Expression of cIAP1, a target for 11q22 amplification, correlates with resistance of cervical cancers to radiotherapy. Cancer Res. 62, 4860-4866.

Jansen B, and Zangemeister-Wittke U. (2002) Antisense therapy for cancer—the time of truth. Lancet Oncol. 3, 672-683.

Kallio M J, Nieminen M, and Eriksson J E. (2001) Human inhibitor of apoptosis protein (IAP) survivin participates in regulation of chromosome segregation and mitotic exit. FASEB J. 15, 2721-2723.

Kanwar J R, Shen W P, Kanwar R K, Berg R W, and Krissansen G W. (2001) Effects of survivin antagonists on growth of established tumors and B7-1 immunogene therapy. J Natl Cancer Inst. 93, 1541-1552.

Kasof G M, and Gomes B C. (2001) Livin, a novel inhibitor of apoptosis protein family member. J Biol Chem. 276, 3238-3246.

Kunath T, Gish G, Lickert H, Jones N, Pawson T, and Rossant J. (2003) Transgenic RNA interference in ES cell-derived embryos recapitulates a genetic null phenotype. Nat Biotechnol. 21, 559-561.

LaCasse E C, Baird S, Korneluk R G, and MacKenzie A E. (1998) The inhibitors of apoptosis (IAPs) and their emerging role in cancer. Oncogene. 17, 3247-3259.

Lens S M, Wolthuis R M, Klompmaker R, Kauw J, Agami R, Brummelkamp T, Kops G, and Medema R H. (2003) Survivin is required for a sustained spindle checkpoint arrest in response to lack of tension. EMBO J. 22, 2934-2947.

Lewis D L, Hagstrom J E, Loomis A G, Wolff J A, and Herweijer H. (2002) Efficient delivery of siRNA for inhibition of gene expression in postnatal mice. Nat Genet. 32, 107-108.

F, Ackermann E J, Bennett C F, Rothermel A L, Plescia J, Tognin S, Villa A, Marchisio P C, and Altieri D C. (1999) Pleiotropic cell-division defects and apoptosis induced by interference with survivin function. Nat Cell Biol. 1, 461-466.

Li J, Feng Q, Kim J M, Schneiderman D, Liston P, Li M, Vanderhyden B, Faught W, Fung M F, Senterman M, Korneluk R G, and Tsang B K. (2001) Human ovarian cancer and cisplatin resistance: possible role of inhibitor of apoptosis proteins. Endocrinology. 142, 370-380.

Lin H, Chen C, and Chen B D. (2001) Resistance of bone marrow-derived macrophages to apoptosis is associated with the expression of X-linked inhibitor of apoptosis protein in primary cultures of bone marrow cells. Biochem J. 353, 299-306.

Liston P, Roy N, Tamai K, Lefebvre C, Baird S, Cherton-Horvat G, Farahani R, McLean M, Ikeda J E, MacKenzie A, and Korneluk R G. (1996) Suppression of apoptosis in mammalian cells by NAIP and a related family of IAP genes. Nature. 379, 349-353.

Liston P, Fong W G, Kelly N L, Toji S, Miyazaki T, Conte D, Tamai K, Craig C G, McBurney M W, and Korneluk R G. (2001) Identification of XAF1 as an antagonist of XIAP anti-Caspase activity. Nat Cell Biol. 3, 128-133.

Liu H, Ruskon-Fourmestraux A, Lavergne-Slove A, Ye H, Molina T, Bouhnik Y, Hamoudi R A, Diss T C, Dogan A, Megraud F, Rambaud J C, Du M Q, and Isaacson P G. (2001) Resistance of t(11; 18) positive gastric mucosa-associated lymphoid tissue lymphoma to Helicobacter pylori eradication therapy. Lancet. 357, 39-40.

Martinez L A, Naguibneva I, Lehrmann H, Vervisch A, Tchenio T, Lozano G, and Harel-Bellan A (2002) Synthetic small inhibiting RNAs: efficient tools to inactivate oncogenic mutations and restore p53 pathways. Proc Natl Acad Sci USA 99: 14849-14854

McCaffrey A P, Meuse L, Pham T T, Conklin D S, Hannon G J, and Kay M A. (2002) RNA interference in adult mice. Nature. 418, 38-39.

McManus M T, and Sharp P A (2002) Gene silencing in mammals by small interfering RNAs. Nature Rev Genet 3, 737-747.

Mesri M, Morales-Ruiz M, Ackermann E J, Bennett C F, Pober J S, Sessa W C, and Altieri D C. (2001) Suppression of vascular endothelial growth factor-mediated endothelial cell protection by survivin targeting. Am J Pathol. 158, 1757-1765.

Miller V M, Xia H, Marrs G L, Gouvion C M, Lee G, Davidson B L, and Paulson H L. (2003) Allele-specific silencing of dominant disease genes. Proc Natl Acad Sci U S A. 100, 7195-7200.

Miranda M B, Dyer K F, Grandis J R, and Johnson D E. (2003) Differential activation of apoptosis regulatory pathways during monocytic vs granulocytic differentiation: a requirement for Bcl-X(L)and XIAP in the prolonged survival of monocytic cells. Leukemia. 17, 390-400.

Hybridon and Aegera collaborate to develop antisense drug. (2002) Expert Rev Anticancer Ther. 2, 483-484.

Olie R A, Simoes-Wust A P, Baumann B, Leech S H, Fabbro D, Stahel R A, and Zangemeister-Wittke U. (2000) A novel antisense oligonucleotide targeting survivin expression induces apoptosis and sensitizes lung cancer cells to chemotherapy. Cancer Res. 60, 2805-2809.

Paddison P J, Caudy A A, Bernstein E, Hannon G J, and Conklin D S. (2002a) Short hairpin RNAs (shRNAs) induce sequence-specific silencing in mammalian cells. Genes Dev. 16, 948-958.

Paddison P J, Caudy A A, and Hannon G J. (2002b) Stable suppression of gene expression by RNAi in mammalian cells. Proc Natl Acad Sci U S A. 99, 1443-1448.

Paddison P J, and Hannon G J. (2002) RNA interference: the new somatic cell genetics? Cancer Cell. 2, 17-23.

Pennati M, Colella G, Folini M, Citti L, Daidone M G, and Zaffaroni N. (2002) Ribozyme-mediated attenuation of survivin expression sensitizes human melanoma cells to cisplatin-induced apoptosis. J Clin Invest. 109, 285-286.

Perrelet D, Ferri A, Liston P, Muzzin P, Korneluk R G, and Kato A C. (2002) IAPs are essential for GDNF-mediated neuroprotective effects in injured motor neurons in vivo. Nat Cell Biol. 4, 175-179.

Pirollo K F, Rait A, Sleer L S, and Chang E H (2003) Antisense therapeutics: from theory to clinical practice. Pharmacol Therapeut 99, 55-77.

Roy N, Mahadevan M S, McLean M, Shutler G, Yaraghi Z, Farahani R, et al (1995) The gene for neuronal apoptosis inhibitory protein is partially deleted in individuals with spinal muscular atrophy. Cell 80, 167-178.

Roy N, Mahadevan M S, McLean M, Shutler G, Yaraghi Z, Farahani R, et al (1995) The gene for neuronal apoptosis inhibitory protein is partially deleted in individuals with spinal muscular atrophy. Cell 80, 167-178.

Roy N, Deveraux Q L, Takahashi R, Salvesen G S, and Reed J C. (1997) The c-IAP-1 and c-IAP-2 proteins are direct inhibitors of specific caspases. EMBO J. 16, 6914-6925.

Rubinson D A, Dillon C P, Kwiatkowski A V, Sievers C, Yang L, Kopinja J, Rooney D L, Ihrig M M, McManus M T, Gertler F B, Scott M L, and Van Parijs L. (2003) A lentivirus-based system to functionally silence genes in primary mammalian cells, stem cells and transgenic mice by RNA interference. Nat Genet. 33, 401-406.

Salvesen G S, and Duckett C S. (2002) IAP proteins: blocking the road to death's door. Nat Rev Mol Cell Biol. 3, 401-410.

Sasaki H, Sheng Y, Kotsuji F, and Tsang B K. (2000) Down-regulation of X-linked inhibitor of apoptosis protein induces apoptosis in chemoresistant human ovarian cancer cells. Cancer Res. 60, 5659-5666.

Shankar S L, Mani S, O'Guin K N, Kandimalla E R, Agrawal S, and Shafit-Zagardo B. (2001) Survivin inhibition induces human neural tumor cell death through caspase-independent and -dependent pathways. J Neurochem. 79, 426-436.

Shen C, Buck A, Polat B, Schmid-Kotsas A, Matuschek C, Gross H J, Bachem M, and Reske S N. (2003) Triplex-forming oligodeoxynucleotides targeting survivin inhibit proliferation and induce apoptosis of human lung carcinoma cells. Cancer Gene Ther. 10, 403-410.

Shuey D J, McCallus D E, and Giordano T (2002) RNAi: gene-silencing in therapeutic intervention. Drug Discov Today 7, 1040-1046

Sohail M, Doran G, Riedemann J, Macaulay V, and Southern E M. (2003) A simple and cost-effective method for producing small interfering RNAs with high efficacy. Nucleic Acids Res. 31, e38.

Sommer K W, Schamberger C J, Schmidt G E, Sasgary S, and Cemi C. (2003) Inhibitor of apoptosis protein (IAP) survivin is upregulated by oncogenic c-H-Ras. Oncogene. 22, 4266-4280.

Song E, Lee S K, Wang J, Ince N, Ouyang N, Min J, Chen J, and Shankar P, Lieberman J. (2003) RNA interference targeting Fas protects mice from fulminant hepatitis. Nat Med. 9, 347-351.

Sorensen D R, Leirdal M, and Sioud M. (2003) Gene silencing by systemic delivery of synthetic siRNAs in adult mice.J Mol Biol. 327, 761-766.

Spalding A C, Jotte R M, Scheinman R I, Geraci M W, Clarke P, Tyler K L, and Johnson G L. (2002) TRAIL and inhibitors of apoptosis are opposing determinants for NF-kappaB-dependent, genotoxin-induced apoptosis of cancer cells. Oncogene. 21, 260-271.

Speliotes E K, Uren A, Vaux D, and Horvitz H R. (2000) The survivin-like C. elegans BIR-1 protein acts with the Aurora-like kinase AIR-2 to affect chromosomes and the spindle midzone. Mol Cell. 6, 211-223.

Stein C A. (2001) The experimental use of antisense oligonucleotides: a guide for the perplexed. J Clin Invest.108, 641-644.

Stein P, Svoboda P, and Schultz R M. (2003) Transgenic RNAi in mouse oocytes: a simple and fast approach to study gene function. Dev Biol. 256, 187-193.

Stennicke H R, Ryan C A, and Salvesen G S (2002) Reprieval from execution: the molecular basis of caspase inhibition. Trends Biochem Sci 27, 94-101.

Stewart S A, Dykxhoorn D M, Palliser D, Mizuno H, Yu E Y, An D S, Sabatini D M, Chen I S, Hahn W C, Sharp P A, Weinberg R A, and Novina C D. (2003) Lentivirus-delivered stable gene silencing by RNAi in primary cells. RNA. 9, 493-501.

Tamm I, Kornblau S M, Segall H, Krajewski S, Welsh K, Kitada S, Scudiero D A, Tudor G, Qui Y H, Monks A, Andreeff M, and Reed J C. (2000) Expression and prognostic significance of IAP-family genes in human cancers and myeloid leukemias. Clin Cancer Res. 6, 1796-1803.

Thornberry N A, and Lazebnik Y. (1998) Caspases: enemies within. Science. 281, 1312-1316.

Tiscomia G, Singer O, Ikawa M, and Verna I M. (2003) A general method for gene knockdown in mice by using lentiviral vectors expressing small interfering RNA. Proc Natl Acad Sci U S A.100, 1844-1848.

Troy C M, Rabacchi S A, Hohl J B, Angelastro J M, Greene L A, and Shelanski M L. (2001) Death in the balance: alternative participation of the caspase-2 and -9 pathways in neuronal death induced by nerve growth factor deprivation. J Neurosci. 21, 5007-5016.

Uren A G, Wong L, Pakusch M, Fowler K J, Burrows F J, Vaux D L, and Choo K H. (2000) Survivin and the inner centromere protein INCENP show similar cell-cycle localization and gene knockout phenotype. Curr Biol. 10, 1319-1328.

van de Wetering M, Oving I, Muncan V, Pon Fong M T, Brantjes H, van Leenen D, Holstege F C, Brummelkamp T R, Agami R, and Clevers H. (2003) Specific inhibition of gene expression using a stably integrated, inducible small-interfering-RNA vector. EMBO Rep. 4, 609-615.

Vickers T A, Koo S, Bennett C F, Crooke S T, Dean N M, and Baker B F. (2003) Efficient reduction of target RNAs by small interfering RNA and RNAse H-dependent antisense agents. A comparative analysis. J Biol Chem. 278, 7108-7118.

Wang J, Tekle E, Oubrahim H, Mieyal J J, Stadtman E R, and Chock P B. (2003) Stable and controllable RNA interference: Investigating the physiological function of glutathionylated actin. Proc Natl Acad Sci U S A. 100, 5103-5106.

Wiese S, Digby M R, Gunnersen J M, Gotz R, Pei G, Holtmann B, Lowenthal J, and Sendtner M. (1999) The anti-apoptotic protein ITA is essential for NGF-mediated survival of embryonic chick neurons. Nat Neurosci. 2, 978-983.

Wilda M, Fuchs U, Wossmann W, and Borkhardt A (2002) Killing of leukemic cells with a BCR/ABL fusion gene by RNA interference (RNAi). Oncogene 21, 5716-5724.

Williams N S, Gaynor R B, Scoggin S, Verma U, Gokaslan T, Simmang C, Fleming J, Tavana D, Frenkel E, and Becerra C. (2003) Identification and validation of genes involved in the pathogenesis of colorectal cancer using cDNA microarrays and RNA interference. Clin Cancer Res. 9, 931-946.

Xia C, Xu Z, Yuan X, Uematsu K, You L, Li K, Li L, McCormick F, and Jablons D M. (2002) Induction of apoptosis in mesothelioma cells by antisurvivin oligonucleotides. Mol Cancer Ther. 1, 687-694.

Xia H, Mao Q, Paulson H L, and Davidson B L. (2002) siRNA-mediated gene silencing in vitro and in vivo. Nat Biotechnol. 20, 1006-1010.

Xu Y, Zhang H Y, Thormeyer D, Larsson O, Du Q, Elmen J, Wahlestedt C, and Liang Z. (2003) Effective small interfering RNAs and phosphorothioate antisense DNAs have different preferences for target sites in the luciferase mRNAs. Biochem Biophys Res Commun. 306, 712-717.

Yamamoto T, Manome Y, Nakamura M, and Tanigawa N. (2002) Downregulation of survivin expression by induction of the effector cell protease receptor-1 reduces tumor growth potential and results in an increased sensitivity to anticancer agents in human colon cancer. Eur J Cancer. 38, 2316-2324.

Yu D, Zhu F-G, Bhagat L, Wang H, Kandimalla E K, Zhang R, and Agrawal S (2002) Potent CpG oligonucleotides containing phosphodiester linkages: in vitro and in vivo immunostimulatory properties. Biochem Biophys Res Commun 297, 83-90

Yu D, Zhu F-G, Bhagat L, Wang H, Kandimalla E K, Zhang R, and Agrawal S (2002) Potent CpG oligonucleotides containing phosphodiester linkages: in vitro and in vivo immunostimulatory properties. Biochem Biophys Res Commun 297, 83-90

Yu J Y, DeRuiter S L, and Turner D L (2002) RNA interference by expression of short-interfering RNAs and hairpin RNAs in mammalian cells. Proc Natl Acad Sci U S A. 99, 6047-6052.

Zender L, Hutker S, Liedtke C, Tillmann H L, Zender S, Mundt B, Waltemathe M, Gosling T, Flemming P, Malek N P, Trautwein C, Manns M P, Kuhnel F, and Kubicka S. (2003) Caspase 8 small interfering RNA prevents acute liver failure in mice. Proc Natl Acad Sci U S A.100, 7797-7802.

Zhang H G, Huang N, Liu D, Bilbao L, Zhang X, Yang P, Zhou T, Curiel D T, and Mountz J D. (2000) Gene therapy that inhibits nuclear translocation of nuclear factor kappaB results in tumor necrosis factor alpha-induced apoptosis of human synovial fibroblasts. Arthritis Rheum. 43, 1094-1105.

Zhou W, and Agrawal S. (1998) Mixed-backbone oligonucleotides as second-generation antisense agents with reduced phosphorothioate-related side effects. Bioorg Med Chem Lett. 8, 3269-3274.

1. A substantially pure nucleobase oligomer comprising a duplex comprising at least eight but no more than thirty consecutive nucleobases corresponding to XIAP (SEQ ID NO: 21), HIAP-1 (SEQ ID NO: 53), or HIAP-2 (SEQ ID NO: 47), wherein said duplex is capable of reducing expression of an IAP polypeptide in a cell.

2. The oligomer of claim 1, wherein said duplex comprises a first domain comprising between 21 and 29 nucleobases and a second domain that hybridizes to said first domain under physiological conditions, wherein said first and second domains are connected by a single stranded loop.

3. The oligomer of claim 2, wherein said loop consists of between 6 and 12 nucleobases.

4. The oligomer of claim 2, wherein said loop consists of 8 nucleobases.

5. The oligomer of claim 1, wherein said duplex is selected from the group consisting of SEQ ID NOs: 32-36, and said oligomer reduces expression of XIAP.

6. The oligomer of claim 1, wherein said duplex is selected from the group consisting of SEQ ID NOs: 42-46, and said oligomer reduces expression of HIAP-2.

7. A nucleobase oligomeric complex comprising paired sense and antisense strands, wherein said complex comprises between eight and thirty consecutive nucleobases corresponding to a sequence of any one of XIAP (SEQ ID NO: 21), HIAP-1 (SEQ ID NO: 53), and HIAP-2 (SEQ ID NO: 47), and said complex is capable of reducing expression of an IAP polypeptide in a cell.

8. The complex of claim 7, wherein said complex comprises any one of SEQ ID NOs: 1-31.

9. The complex of claim 7, wherein said complex comprises any one of SEQ ID NOs: 37-41 and 54-63.

10. The complex of claim 8, wherein said complex is dsRNA.

11. The complex of claim 8, wherein said complex comprises at least one modification.

12. The complex of claim 11, wherein said complex comprises at least two modifications.

13. The complex of claim 11, wherein said modification is a modified sugar moiety, nucleobase, or internucleoside linkage.

14. The complex of claim 13, wherein said modification is a modified internucleoside linkage selected from the group consisting of phosphorothioate, methylphosphonate, phosphotriester, phosphorodithioate, and phosphoselenate linkages.

15. The complex of claim 13, wherein said modification is a modified sugar moiety.

16. The complex of claim 13, wherein said modified sugar moiety is a 2′-O-methyl group or a 2′-O-methoxyethyl group.

17. The complex of claim 13, wherein said modification is a modified nucleobase.

18. The complex of claim 17, wherein said modified nucleobase is 5-methyl cytosine.

19. The complex of claim 17, wherein said modified nucleobase is a chimeric nucleobase oligomer.

20. The complex of claim 19, wherein said chimeric nucleobase oligomer comprises a plurality of RNA residues.

21. The complex of claim 20, wherein said RNA residues are linked together by phosphorothioate linkages.

22. An expression vector encoding a nucleobase oligomer of claim 1 or nucleobase oligomeric complex of claim 7.

23. The expression vector of claim 22, wherein a nucleic acid sequence encoding said nucleobase oligomer or nucleobase oligomeric complex is operably linked to a promoter.

24. The expression vector of claim 23, wherein said promoter is the U6 PolIII promoter, or the polymerase III H1 promoter.

25. A cell comprising the expression vector of claim 21.

26. The cell of claim 25, wherein said cell is a transformed human cell that stably expresses said expression vector.

27. The cell of claim 25, wherein said cell is in vivo.

28. The cell of claim 27, wherein said cell is a human cell.

29. The cell of claim 28, wherein said cell is a neoplastic cell.

30. The cell of claim 27, wherein said cell is in a human.

31. A method of enhancing apoptosis in a cell, said method comprising contacting said cell with an apoptosis-enhancing amount of a nucleobase oligomer of claim 1 or an oligomeric complex of claim 8.

32. The method of claim 31, wherein said cell is in vitro.

33. The method of claim 31, wherein said cell is in vivo.

34. The method of claim 31, wherein said cell is a human cell.

35. The method of claim 34, wherein said cell is a cancer cell

36. The method of claim 33, wherein said cell is from a human.

37. A method of enhancing apoptosis in a cell, said method comprising contacting said cell with (i) a nucleobase oligomer of claim 1 or an oligomeric complex of claim 8; and (ii) a chemotherapeutic agent in amounts that together are sufficient to enhance apoptosis in said cell.

38. The method, according to claim 37, in which the chemotherapeutic agent is selected from the agents listed on Table 5.

39. A method of enhancing apoptosis in a cell, said method comprising contacting said cell with (i) a nucleobase oligomer of claim 1 or an oligomeric complex of claim 8; and (ii) a chemosensitizing agent in amounts that together are sufficient to enhance apoptosis in said cell.

40. A method of treating a proliferative disease in an individual in need thereof, said method comprising administering to said individual a nucleobase oligomer of claim 1 or an oligomeric complex of claim 8 in an amount effective to treat said individual.

41. A method of treating a proliferative disease in an individual in need thereof, said method comprising administering to said individual (i) a nucleobase oligomer of claim 1 or an oligomeric complex of claim 8; and (ii) a chemotherapeutic agent in amounts sufficient to treat said individual.

42. A method of treating a proliferative disease in an individual in need thereof, said method comprising administering to said individual (i) a nucleobase oligomer of claim 1 or an oligomeric complex of claim 8; and (ii) a chemosensitizing agent in amounts sufficient to treat said individual.

43. The method of any one of claims 40-42, wherein said animal is a human.

44. The method of any one of claims 40-42, wherein said proliferative disorder is cancer.

45. The method of claim 44, wherein said cancer is selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

46. A kit comprising:
(i) a nucleobase oligomer of claim 1 or an oligomeric complex of claim 8; and
(ii) instructions to administer said nucleobase oligomer or oligomeric complex to an individual having a proliferative disease in an amount sufficient to treat said proliferative disease.

47. A kit comprising:
(i) a nucleobase oligomer of claim 1 or an oligomeric complex of claim 8; and
(ii) instructions to administer said nucleobase oligomer or oligomeric complex and a chemotherapeutic agent to an individual having a proliferative disease in amounts sufficient to treat said proliferative disease.

48. A kit comprising:
(i) a nucleobase oligomer of claim 1 or an oligomeric complex of claim 8; and
(ii) instructions to administer said nucleobase oligomer or oligomeric complex and a chemosensitizing agent to an individual having a proliferative disease in amounts sufficient to treat said proliferative disease.


SEQUENCE LISTING (65 sequences)

SEQ ID NO: 1 - DNA, 21 bp, Artificial Sequence
  misc_feature 14, 15: n = T or U
  based on Homo sapiens Each nucleobase may be part of a ribonucleotide, deoxyribonucleotide, or nucleotide analog
aacaaggagcagcnngcaaga

SEQ ID NO: 2 - DNA, 21 bp, Artificial Sequence
  misc_feature 15, 16, 17, 20: n = T or U
  based on Homo sapiens Each nucleobase may be part of a ribonucleotide, deoxyribonucleotide, or nucleotide analog
aacacaggcgacacnnnccna

SEQ ID NO: 3 - DNA, 21 bp, Artificial Sequence
  misc_feature 11, 12, 21: n = T or U
  based on Homo sapiens Each nucleobase may be part of a ribonucleotide, deoxyribonucleotide, or nucleotide analog
aaggagcagcnngcaagagcn

SEQ ID NO: 4 - DNA, 21 bp, Artificial Sequence
  misc_feature 5, 6, 8, 11, 14, 18, 21: n = T or U
  based on Homo sapiens Each nucleobase may be part of a ribonucleotide, deoxyribonucleotide, or nucleotide analog
aaccnngngancgngccnggn

SEQ ID NO: 5 - DNA, 21 bp, Artificial Sequence
  misc_feature 5, 8, 9, 10, 14, 16: n = T or U
  based on Homo sapiens Each nucleobase may be part of a ribonucleotide, deoxyribonucleotide, or nucleotide analog
aaagngcnnncacngnggagg

SEQ ID NO: 6 - DNA, 21 bp, Artificial Sequence
  misc_feature 4, 7, 8, 9, 13, 15: n = T or U
  based on Homo sapiens Each nucleobase may be part of a ribonucleotide, deoxyribonucleotide, or nucleotide analog
aagngcnnncacngnggagga

SEQ ID NO: 7 - DNA, 21 bp, Artificial Sequence
  misc_feature 4, 7, 8, 19: n = T or U
  based on Homo sapiens Each nucleobase may be part of a ribonucleotide, deoxyribonucleotide, or nucleotide analog
aacngannggaagcccagnga

SEQ ID NO: 8 - DNA, 21 bp, Artificial Sequence
  misc_feature 4, 7, 12, 14, 17: n = T or U
  based on Homo sapiens Each nucleobase may be part of a ribonucleotide, deoxyribonucleotide, or nucleotide analog
aagngcnggacncnacnacac

SEQ ID NO: 9 - DNA, 21 bp, Artificial Sequence
  misc_feature 8, 9, 20: n = T or U
  based on Homo sapiens Each nucleobase may be part of a ribonucleotide, deoxyribonucleotide, or nucleotide analog
aagacccnngggaacaacang

SEQ ID NO: 10 - DNA, 21 bp, Artificial Sequence
  misc_feature 8, 9, 16, 19: n = T or U
  based on Homo sapiens Each nucleobase may be part of a ribonucleotide, deoxyribonucleotide, or nucleotide analog
aagagagnnagcaagngcngg

SEQ ID NO: 11 - DNA, 21 bp, Artificial Sequence
  misc_feature 4, 13, 15, 18: n = T or U
  based on Homo sapiens Each nucleobase may be part of a ribonucleotide, deoxyribonucleotide, or nucleotide analog
aacnggccagacnangcncac

SEQ ID NO: 12 - DNA, 21 bp, Artificial Sequence
  misc_feature 4, 7, 8, 9, 16: n = T or U
  based on Homo sapiens Each nucleobase may be part of a ribonucleotide, deoxyribonucleotide, or nucleotide analog
aagnccnnncagaacnggcca

SEQ ID NO: 13 - DNA, 21 bp, Artificial Sequence
  misc_feature 5, 8, 9, 10, 17: n = T or U
  based on Homo sapiens Each nucleobase may be part of a ribonucleotide, deoxyribonucleotide, or nucleotide analog
aaagnccnnncagaacnggcc

SEQ ID NO: 14 - DNA, 21 bp, Artificial Sequence
  misc_feature 3, 6, 16, 18: n = T or U
  based on Homo sapiens Each nucleobase may be part of a ribonucleotide, deoxyribonucleotide, or nucleotide analog
aanagngccacgcagncnaca

SEQ ID NO: 15 - DNA, 21 bp, Artificial Sequence
  misc_feature 4, 7, 17, 19: n = T or U
  based on Homo sapiens Each nucleobase may be part of a ribonucleotide, deoxyribonucleotide, or nucleotide analog
aaanagngccacgcagncnac

SEQ ID NO: 16 - DNA, 21 bp, Artificial Sequence
  misc_feature 4, 7, 10, 13, 15, 16, 17: n = T or U
  based on Homo sapiens Each nucleobase may be part of a ribonucleotide, deoxyribonucleotide, or nucleotide analog
aagnggnagnccngnnncagc

SEQ ID NO: 17 - DNA, 21 bp, Artificial Sequence
  misc_feature 7, 8, 15, 17: n = T or U
  based on Homo sapiens Each nucleobase may be part of a ribonucleotide, deoxyribonucleotide, or nucleotide analog
aagcagnngacaagngnccca

SEQ ID NO: 18 - DNA, 21 bp, Artificial Sequence
  misc_feature 4, 6, 11, 13, 16: n = T or U
  based on Homo sapiens Each nucleobase may be part of a ribonucleotide, deoxyribonucleotide, or nucleotide analog
aagngncccangngcnacaca

SEQ ID NO: 19 - DNA, 21 bp, Artificial Sequence
  misc_feature 1, 10, 14, 18: n = T or U
  based on Homo sapiens Each nucleobase may be part of a ribonucleotide, deoxyribonucleotide, or nucleotide analog
nccaagaaanccanccanggc

SEQ ID NO: 20 - DNA, 21 bp, Artificial Sequence
  misc_feature 9, 13, 17: n = T or U
  based on Homo sapiens Each nucleobase may be part of a ribonucleotide, deoxyribonucleotide, or nucleotide analog
ccaagaaanccanccanggca

SEQ ID NO: 21 - DNA, 1494 bp, Homo sapiens
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaa

SEQ ID NO: 22 - DNA, 21 bp, Artificial Sequence
  misc_feature 8, 9, 16, 19: n = T or U
  based on Homo sapiens Each nucleobase may be part of a ribonucleotide, deoxyribonucleotide, or nucleotide analog
aagagagnnagcaagngcngg

SEQ ID NO: 23 - DNA, 21 bp, Artificial Sequence
  misc_feature 4, 7, 12, 14, 17: n = T or U
  based on Homo sapiens Each nucleobase may be part of a ribonucleotide, deoxyribonucleotide, or nucleotide analog
aagngcnggacncnacnacac

SEQ ID NO: 24 - DNA, 21 bp, Artificial Sequence
  misc_feature 5, 6, 8, 11, 14, 18, 21: n = T or U
  based on Homo sapiens Each nucleobase may be part of a ribonucleotide, deoxyribonucleotide, or nucleotide analog
aaccnngngancgngccnggn

SEQ ID NO: 25 - DNA, 21 bp, Artificial Sequence
  misc_feature 15, 16, 17, 20: n = T or U
  based on Homo sapiens Each nucleobase may be part of a ribonucleotide, deoxyribonucleotide, or nucleotide analog
aacacaggcgacacnnnccna

SEQ ID NO: 26 - DNA, 21 bp, Artificial Sequence
  misc_feature 14, 15: n = T or U
  based on Homo sapiens Each nucleobase may be part of a ribonucleotide, deoxyribonucleotide, or nucleotide analog
aacaaggagcagcnngcaaga

SEQ ID NO: 27 - DNA, 21 bp, Artificial Sequence
  misc_feature 11, 12, 21: n = T or U
  based on Homo sapiens Each nucleobase may be part of a ribonucleotide, deoxyribonucleotide, or nucleotide analog
aaggagcagcnngcaagagcn

SEQ ID NO: 28 - DNA, 21 bp, Artificial Sequence
  misc_feature 5, 8, 9, 10, 14, 16: n = T or U
  based on Homo sapiens Each nucleobase may be part of a ribonucleotide, deoxyribonucleotide, or nucleotide analog
aaagngcnnncacngnggagg

SEQ ID NO: 29 - DNA, 21 bp, Artificial Sequence
  misc_feature 4, 7, 8, 9, 13, 15: n = T or U
  based on Homo sapiens Each nucleobase may be part of a ribonucleotide, deoxyribonucleotide, or nucleotide analog
aagngcnnncacngnggagga

SEQ ID NO: 30 - DNA, 21 bp, Artificial Sequence
  misc_feature 4, 7, 8, 19: n = T or U
  based on Homo sapiens Each nucleobase may be part of a ribonucleotide, deoxyribonucleotide, or nucleotide analog
aacngannggaagcccagnga

SEQ ID NO: 31 - DNA, 21 bp, Artificial Sequence
  misc_feature 8, 9, 20: n = T or U
  based on Homo sapiens Each nucleobase may be part of a ribonucleotide, deoxyribonucleotide, or nucleotide analog
aagacccnngggaacaacang

SEQ ID NO: 32 - DNA, 93 bp, Artificial Sequence
  misc_feature 1, 2, 4, 33, 35, 37, 40, 41, 48, 50, 56, 57, 60, 63, 66,75, 78, 80, 85, 87, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccgcaccgcacggnancnccnncaccagnangaagcnnganacnggngaggggganacngngcggngnnnnnnn

SEQ ID NO: 33 - DNA, 93 bp, Artificial Sequence
  misc_feature 1, 2, 4, 23, 26, 30, 32, 34, 41, 49, 50, 56, 57, 62, 64,66, 69, 71, 74, 78, 81, 84, 85, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccgnaangacngngnagcacangggacacnngaagcnngaggngncncangngcngcanagncannacnnnnnn

SEQ ID NO: 34 - DNA, 93 bp, Artificial Sequence
  misc_feature 1, 2, 4, 23, 25, 26, 28, 41, 49, 50, 56, 57, 63, 64, 66,69, 72, 75, 76, 79, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccgngnncngaccaggcacgancacaaggnngaagcnngagccnngngancgngcnnggncggagcacnnnnnn

SEQ ID NO: 35 - DNA, 93 bp, Artificial Sequence
  misc_feature 1, 2, 4, 24, 25, 28, 33, 35, 37, 40, 42, 44, 47, 49, 56,57, 59, 61, 63, 73, 75, 78, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccggnnccncgggnananggngncngananagaagcnngngnancagacaccgnanacncgagggaccnnnnnn

SEQ ID NO: 36 - DNA, 93 bp, Artificial Sequence
  misc_feature 1, 2, 4, 24, 26, 29, 33, 34, 36, 37, 39, 42, 43, 46, 49,56, 57, 61, 65, 69, 77, 78, 81, 84, 88, 89, 90, 91, 92,93: n = T or U
  based on Homo sapiens Each nucleobase may be part of a ribonucleotide, deoxyribonucleotide, or nucleotide analog
nngnggaaaggacgaaacaccggnancancaanncnncnagnnagnganggaagcnngcgncgcngacnaggagaannganganaccnnnnnn

SEQ ID NO: 37 - DNA, 29 bp, Artificial Sequence
  misc_feature 2, 5, 8, 17, 22, 27: n = T or U
  based on Homo sapiens Each nucleobase may be part of a ribonucleotide, deoxyribonucleotide, or nucleotide analog
anacnggngaaggaganaccgngcggngc

SEQ ID NO: 38 - DNA, 29 bp, Artificial Sequence
  misc_feature 4, 6, 11, 13, 16, 23, 26, 27: n = T or U
  based on Homo sapiens Each nucleobase may be part of a ribonucleotide, deoxyribonucleotide, or nucleotide analog
aagngncccangngcnacacagncannac

SEQ ID NO: 39 - DNA, 29 bp, Artificial Sequence
  misc_feature 5, 6, 8, 11, 14, 18, 21: n = T or U
  based on Homo sapiens Each nucleobase may be part of a ribonucleotide, deoxyribonucleotide, or nucleotide analog
aaccnngngancgngccnggncagaacac

SEQ ID NO: 40 - DNA, 29 bp, Artificial Sequence
  misc_feature 1, 3, 5, 15, 17: n = T or U
  based on Homo sapiens Each nucleobase may be part of a ribonucleotide, deoxyribonucleotide, or nucleotide analog
nanancagacaccananacccgaggaacc

SEQ ID NO: 41 - DNA, 29 bp, Artificial Sequence
  misc_feature 3, 7, 11, 19, 20, 23, 26: n = T or U
  based on Homo sapiens Each nucleobase may be part of a ribonucleotide, deoxyribonucleotide, or nucleotide analog
cancacnaacnagaagaannganganacc

SEQ ID NO: 42 - DNA, 93 bp, Artificial Sequence
  misc_feature 1, 2, 4, 23, 26, 28, 30, 31, 35, 36, 38, 41, 43, 44, 47,49, 50, 56, 57, 63, 64, 70, 72, 75, 82, 88, 89, 90, 91,92, 93: n = T or U
  based on Homo sapiens Each nucleobase may be part of a ribonucleotide, deoxyribonucleotide, or nucleotide analog
nngnggaaaggacgaaacaccgngcnancnnccannanacncnnaangnngaagcnngagcannaggagnanaangggaggnagcacnnnnnn

SEQ ID NO: 43 - DNA, 93 bp, Artificial Sequence
  misc_feature 1, 2, 4, 23, 32, 37, 38, 41, 42, 44, 50, 56, 57, 64, 66,69, 76, 78, 79, 81, 82, 85, 87, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccgnacgaacagnacccnngannanaccccngaagcnngaggggnangancaggggnanngnncgnannnnnnn

SEQ ID NO: 44 - DNA, 93 bp, Artificial Sequence
  misc_feature 1, 2, 4, 28, 31, 37, 42, 50, 56, 57, 61, 63, 64, 66, 69,70, 75, 76, 84, 85, 88, 89, 90, 91, 92, 93: n = T or U
  based on Homo sapiens Each nucleobase may be part of a ribonucleotide, deoxyribonucleotide, or nucleotide analog
nngnggaaaggacgaaacaccgcaagcngcncaggcngaacnggaacacngaagcnngagngnncnagnncggcnngggcagcnngcnnnnnn

SEQ ID NO: 45 - DNA, 93 bp, Artificial Sequence
  misc_feature 1, 2, 4, 23, 26, 29, 30, 34, 38, 40, 46, 47, 50, 56, 57,60, 61, 66, 68, 73, 74, 76, 78, 81, 85, 88, 89, 90, 91,92, 93: n = T or U
  based on Homo sapiens Each nucleobase may be part of a ribonucleotide, deoxyribonucleotide, or nucleotide analog
nngnggaaaggacgaaacaccgnacncanngcanaacngnaggggnnagngaagcnngannagccncnacagnnangnaangagnacnnnnnn

SEQ ID NO: 46 - DNA, 93 bp, Artificial Sequence
  misc_feature 1, 2, 4, 26, 27, 29, 31, 32, 34, 35, 41, 42, 45, 56, 57,59, 60, 62, 63, 65, 66, 69, 70, 84, 87, 88, 89, 90, 91,92, 93: n = T or U
  based on Homo sapiens Each nucleobase may be part of a ribonucleotide, deoxyribonucleotide, or nucleotide analog
nngnggaaaggacgaaacaccgccanncngnncnnccgaannaangacaagaagcnngnngnnannaanncggaggagcagaanggnnnnnnn

SEQ ID NO: 47 - DNA, 1857 bp, Homo sapiens
atgcacaaaactgcctcccaaagacttttcccaggtccctcgtatcaaaacattaagagtataatggaagatagcacgatcttgtcagattggacaaacagcaacaaacaaaaaatgaagtatgacttttcctgtgaactctacagaatgtctacatattcaactttccccgccggggtgcctgtctcagaaaggagtcttgctcgtgctggtttttattatactggtgtgaatgacaaggtcaaatgcttctgttgtggcctgatgctggataactggaaactaggagacagtcctattcaaaagcataaacagctatatcctagctgtagctttattcagaatctggtttcagctagtctgggatccacctctaagaatacgtctccaatgagaaacagttttgcacattcattatctcccaccttggaacatagtagcttgttcagtggttcttactccagccttcctccaaaccctcttaattctagagcagttgaagacatctcttcatcgaggactaacccctacagttatgcaatgagtactgaagaagccagatttcttacctaccatatgtggccattaacttttttgtcaccatcagaattggcaagagctggtttttattatataggacctggagatagggtagcctgctttgcctgtggtgggaagctcagtaactgggaaccaaaggatgatgctatgtcagaacaccggaggcattttcccaactgtccatttttggaaaattctctagaaactctgaggtttagcatttcaaatctgagcatgcagacacatgcagctcgaatgagaacatttatgtactggccatctagtgttccagttcagcctgagcagcttgcaagtgctggtttttattatgtgggtcgcaatgatgatgtcaaatgctttggttgtgatggtggcttgaggtgttgggaatctggagatgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttctaagtaaaatcctgacaactggagagaactataaaacagttaatgatattgtgtcagcacttcttaatgctgaagatgaaaaaagagaagaggagaaggaaaaacaagctgaagaaatggcatcagatgatttgtcattaattcggaagaacagaatggctctctttcaacaattgacatgtgtgcttcctatcctggataatcttttaaaggccaatgtaattaataaacaggaacatgatattattaaacaaaaaacacagatacctttacaagcgagagaactgattgataccatttgggttaaaggaaatgctgcggccaacatcttcaaaaactgtctaaaagaaattgactctacattgtataagaacttatttgtggataagaatatgaagtatattccaacagaagatgtttcaggtctgtcactggaagaacaattgaggaggttgcaagaagaacgaacttgtaaagtgtgtatggacaaagaagtttctgttgtatttattccttgtggtcatctggtagtatgccaggaatgtgccccttctctaagaaaatgccctatttgcaggggtataatcaagggtactgttcgtacatttctctcttaa

SEQ ID NO: 48 - DNA, 29 bp, Artificial Sequence
  misc_feature 5, 6, 12, 14, 17, 24: n = T or U
  based on Homo sapiens Each nucleobase may be part of a ribonucleotide, deoxyribonucleotide, or nucleotide analog
aacannaagagnanaanggaaganagcac

SEQ ID NO: 49 - DNA, 29 bp, Artificial Sequence
  misc_feature 6, 8, 11, 18, 21, 23, 24, 27: n = T or U
  based on Homo sapiens Each nucleobase may be part of a ribonucleotide, deoxyribonucleotide, or nucleotide analog
aggggnanaancaagggnacngnncgnac

SEQ ID NO: 50 - DNA, 29 bp, Artificial Sequence
  misc_feature 3, 5, 6, 11, 12, 18, 26, 27: n = T or U
  based on Homo sapiens Each nucleobase may be part of a ribonucleotide, deoxyribonucleotide, or nucleotide analog
agngnnccagnncagccngagcagcnngc

SEQ ID NO: 51 - DNA, 29 bp, Artificial Sequence
  misc_feature 3, 10, 15, 16, 18, 23, 27: n = T or U
  based on Homo sapiens Each nucleobase may be part of a ribonucleotide, deoxyribonucleotide, or nucleotide analog
acnaaccccnacagnnangcaangagnac

SEQ ID NO: 52 - DNA, 29 bp, Artificial Sequence
  misc_feature 1, 2, 4, 7, 8, 11, 12, 26: n = T or U
  based on Homo sapiens Each nucleobase may be part of a ribonucleotide, deoxyribonucleotide, or nucleotide analog
nngncannaanncggaagaacagaanggc

SEQ ID NO: 53 - DNA, 1815 bp, Homo sapiens
atgaacatagtagaaaacagcatattcttatcaaatttgatgaaaagcgccaacacgtttgaactgaaatacgacttgtcatgtgaactgtaccgaatgtctacgtattccacttttcctgctggggttcctgtctcagaaaggagtcttgctcgtgctggtttctattacactggtgtgaatgacaaggtcaaatgcttctgttgtggcctgatgctggataactggaaaagaggagacagtcctactgaaaagcataaaaagttgtatcctagctgcagattcgttcagagtctaaattccgttaacaacttggaagctacctctcagcctacttttccttcttcagtaacacattccacacactcattacttccgggtacagaaaacagtggatatttccgtggctcttattcaaactctccatcaaatcctgtaaactccagagcaaatcaagaattttctgccttgatgagaagttcctacccctgtccaatgaataacgaaaatgccagattacttacttttcagacatggccattgacttttctgtcgccaacagatctggcacgagcaggcttttactacataggacctggagacagagtggcttgctttgcctgtggtggaaaattgagcaattgggaaccgaaggataatgctatgtcagaacacctgagacattttcccaaatgcccatttatagaaaatcagcttcaagacacttcaagatacacagtttctaatctgagcatgcagacacatgcagcccgctttaaaacattctttaactggccctctagtgttctagttaatcctgagcagcttgcaagtgcgggtttttattatgtgggtaacagtgatgatgtcaaatgcttttgctgtgatggtggactcaggtgttgggaatctggagatgatccatgggttcaacatgccaagtggtttccaaggtgtgagtacttgataagaattaaaggacaggagttcatccgtcaagttcaagccagttaccctcatctacttgaacagctgctatccacatcagacagcccaggagatgaaaatgcagagtcatcaattatccatttggaacctggagaagaccattcagaagatgcaatcatgatgaatactcctgtgattaatgctgccgtggaaatgggctttagtagaagcctggtaaaacagacagttcagagaaaaatcctagcaactggagagaattatagactagtcaatgatcttgtgttagacttactcaatgcagaagatgaaataagggaagaggagagagaaagagcaactgaggaaaaagaatcaaatgatttattattaatccggaagaatagaatggcactttttcaacatttgacttgtgtaattccaatcctggatagtctactaactgccggaattattaatgaacaagaacatgatgttattaaacagaagacacagacgtctttacaagcaagagaactgattgatacgattttagtaaaaggaaatattgcagccactgtattcagaaactctctgcaagaagctgaagctgtgttatatgagcatttatttgtgcaacaggacataaaatatattcccacagaagatgtttcagatctaccagtggaagaacaattgcggagactaccagaagaaagaacatgtaaagtgtgtatggacaaagaagtgtccatagtgtttattccttgtggtcatctagtagtatgcaaagattgtgctccttctttaagaaagtgtcctatttgtaggagtacaatcaagggtacagttcgtacatttctttcatga

SEQ ID NO: 54 - DNA, 29 bp, Artificial Sequence
  misc_feature 4, 5, 7, 10, 12, 17, 19, 26, 28: n = T or U
  based on Homo sapiens Each nucleobase may be part of a ribonucleotide, deoxyribonucleotide, or nucleotide analog
gacnngncangngaacngnaccgaangnc

SEQ ID NO: 55 - DNA, 29 bp, Artificial Sequence
  misc_feature 6, 11, 18, 23, 24, 27: n = T or U
  based on Homo sapiens Each nucleobase may be part of a ribonucleotide, deoxyribonucleotide, or nucleotide analog
aggagnacaancaagggnacagnncgnac

SEQ ID NO: 56 - DNA, 29 bp, Artificial Sequence
  misc_feature 3, 6, 8, 11, 12, 20, 23, 25: n = T or U
  based on Homo sapiens Each nucleobase may be part of a ribonucleotide, deoxyribonucleotide, or nucleotide analog
ccncancnacnngaacagcngcnanccac

SEQ ID NO: 57 - DNA, 29 bp, Artificial Sequence
  misc_feature 10, 13, 16, 18, 20: n = T or U
  based on Homo sapiens Each nucleobase may be part of a ribonucleotide, deoxyribonucleotide, or nucleotide analog
accgaagganaangcnangncagaacacc

SEQ ID NO: 58 - DNA, 29 bp, Artificial Sequence
  misc_feature 1, 8, 17, 18, 20, 25, 28: n = T or U
  based on Homo sapiens Each nucleobase may be part of a ribonucleotide, deoxyribonucleotide, or nucleotide analog
nagcaacnggagagaannanagacnagnc

SEQ ID NO: 59 - DNA, 29 bp, Artificial Sequence
  misc_feature 5, 6, 9, 15, 17, 22, 26: n = T or U
  based on Homo sapiens Each nucleobase may be part of a ribonucleotide, deoxyribonucleotide, or nucleotide analog
gaagnnccnaccccngnccaangaanaac

SEQ ID NO: 60 - DNA, 28 bp, Artificial Sequence
  misc_feature 2, 5, 6, 11, 14, 18, 21, 23, 26: n = T or U
  based on Homo sapiens Each nucleobase may be part of a ribonucleotide, deoxyribonucleotide, or nucleotide analog
gnaannccaanccngganagncnacnaa

SEQ ID NO: 61 - DNA, 29 bp, Artificial Sequence
  misc_feature 1, 3, 6, 9, 14, 19, 21, 22, 28: n = T or U
  based on Homo sapiens Each nucleobase may be part of a ribonucleotide, deoxyribonucleotide, or nucleotide analog
ngnganggnggacncaggngnngggaanc

SEQ ID NO: 62 - DNA, 29 bp, Artificial Sequence
  misc_feature 3, 4, 12, 17, 20, 23, 27: n = T or U
  based on Homo sapiens Each nucleobase may be part of a ribonucleotide, deoxyribonucleotide, or nucleotide analog
canncagaagangcaancangangaanac

SEQ ID NO: 63 - DNA, 29 bp, Artificial Sequence
  misc_feature 3, 4, 8, 9, 16, 17, 24, 28: n = T or U
  based on Homo sapiens Each nucleobase may be part of a ribonucleotide, deoxyribonucleotide, or nucleotide analog
aannccgnnaacaacnnggaagcnaccnc

SEQ ID NO: 64 - DNA, 29 bp, Artificial Sequence
  misc_feature 3, 5, 7, 18, 20, 21, 24, 27: n = T or U
  based on Homo sapiens Each nucleobase may be part of a ribonucleotide, deoxyribonucleotide, or nucleotide analog
ccngncncagaaaggagncnngcncgngc

SEQ ID NO: 65 - DNA, 29 bp, Artificial Sequence
  misc_feature 3, 4, 6, 8, 9, 11, 16, 19, 22, 26: n = T or U
  based on Homo sapiens Each nucleobase may be part of a ribonucleotide, deoxyribonucleotide, or nucleotide analog
gcnncngnngnggccngangcngganaac